(19) United States
(12) Patent Application Publication
(10) Pub. No.: US 2022/0106623 A1
(43) Pub. Date: Apr. 7, 2022

(54) SPATIAL MAPPING OF CELLS AND CELL TYPES IN COMPLEX TISSUES

(71) Applicant: Fred Hutchinson Cancer Research Center, Seattle, WA (US)

(72) Inventor: Jason Bielas, Seattle, WA (US)

(73) Assignee: Fred Hutchinson Cancer Research Center, Seattle, WA (US)

(21) Appl. No.: 17/296,038

(22) PCT Filed: Nov. 21, 2019
(86) PCT No.: PCT/US2019/062628

§ 371 Date: May 21, 2021

Related U.S. Application Data
(60) Provisional application No. 62/770,661, filed on Nov. 21, 2018.

(51) Int. Cl. C12Q 1/6804; G01N 33/574; C12Q 1/686; C12Q 1/6874; C12N 15/10

(52) U.S. Cl. CPC C12Q 1/6804; G01N 33/574; C12Q 1/686; C12Q 1/6874; G01N 2333/70596; C12N 15/1086; G01N 2333/70514; G01N 2333/70517; G01N 2333/70589; C12N 15/1068

(57) ABSTRACT

Systems and methods to tag cells in a tissue sample with spatial identifiers, so that spatial reconstruction of cell locations within a tissue can be achieved after tissue disaggregation are described. The systems and methods allow the control or directing of specific spatial identifiers spatially within or throughout a tissue to individual cells or to a small population of cells so that cell position can be determined by computational deconvolution of the spatial identifiers after tissue disaggregation. The systems and methods can be combined with single cell expression analysis to correlate cell types with cell location within a structure, such as a tumor.

### CROSS-REFERENCE TO RELATED APPLICATION

This application claims priority to U.S. Provisional Patent Application No. 62/770,661 filed Nov. 21, 2018, which is incorporated herein by reference in its entirety as if fully set forth herein.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 27B8719_ST25.txt. The text file is 3.28 KB, was created on November 20, 2019, and is being submitted electronically via EFS-Web.

### FIELD OF THE DISCLOSURE

The current disclosure provides systems and methods to tag cells in a tissue sample with spatial identifiers, so that spatial reconstruction of cell locations within a tissue can be achieved after tissue disaggregation. The systems and methods can be combined with single cell expression analysis to correlate cell types with cell location within a structure, such as a tumor.

### BACKGROUND OF THE DISCLOSURE

An organism is made up of organs and in turn, each organ is made up of tissues. Each tissue is a collection of similar cells that can carry out a particular function of the tissue. Although cells in a given tissue may be similar, there may be differences that are masked when observing all the cells in the tissue in bulk, and these differences are only uncovered when one looks at each individual cell. The differences may include cell shape, cell size, developmental stage of the cell, chromosome structure in each cell, what genes are expressed in each cell, and what products are made or secreted by each cell. Each cell within a tissue is also sensing and communicating with other cells around it, and each cell's behavior may be affected by surrounding cells. Thus, to understand the function of a tissue and to be able to address problems that prevent a tissue from functioning properly, it would be very powerful to not only understand what is happening in a tissue at a single cell level but also be able to know where each cell is spatially within the tissue.

The implications of having a heterogeneous population of cells within a tissue is highlighted in cancer with tumor tissues. A given tumor tissue in an individual can have tumorigenic cells, blood vessels that support tumor growth, immune cells that attack the tumor, cells that provide a structural framework, cells that secrete molecules that provide a structural framework for the tissue, and signaling molecules that are secreted by cells to communicate with one another. Cells within tumor tissue can also behave differently depending on where each cell is spatially within the tumor, as regional differences can have different oxygen levels, different signaling molecules, and different cell types. A needle biopsy is often taken to examine tumors, but the heterogeneity of a tumor can render observation of disease features in the tumor challenging based on the small fraction of cells collected from the biopsy, which would not be representative of the whole tumor.

Furthermore, tumor-infiltrating lymphocytes (TILs) have been shown to directly attack tumor cells in a variety of types of cancer, and multiple independent studies have demonstrated that the presence of TI Ls is strongly correlated with increased survival. However, existing assays and pathology tests to measure TILs are cumbersome, have inherent variability, and are not used for clinical decision-making.

Methods exist to identify individual cell types within a sample based on expression analysis. For example, single-cell RNA-seq (scRNA-seq) can identify the immune cell composition in blood and bone marrow. Moreover, it has been shown that scRNA-seq can quantify intra-population heterogeneity and enable study of cell states and transitions at very high resolution, revealing cell subtypes or gene expression dynamics that are masked in bulk, population-averaged measurements (Zheng et al., Nat Commun. 8: 14049, 2017; US20170260584). Nevertheless, although the scRNA-seq method and other recently developed technologies allow whole-transcriptome sequencing of individual cells to identify cell types, they lack the ability to spatially characterize cells in complex tissues, as this information is lost when the tissue is disaggregated to perform required sequencing.

### SUMMARY OF THE DISCLOSURE

The current disclosure provides systems and methods of cellular spatial mapping, optionally combined with high-throughput single cell transcriptomics to help unravel the biology of tissues, such as tumors. In particular embodiments, the systems and methods can be used to spatially map tumor-infiltrating immune cells, such as tumor-infiltrating lymphocytes (TILs). This map information can be used, for example, to assess the clinical utility of characterizing TILs in relation to their position within a tumor. In particular embodiments, the systems and methods of the present disclosure can be used to comprehensively characterize the immune cell composition of non-small cell lung cancer, where the response rate to immune checkpoint inhibitor therapy is only 20%.

Particular embodiments achieve spatial mapping by tagging cells in a biological sample with one or more spatial identifiers, such as nucleic acid barcodes. In particular embodiments, the spatial identifier barcodes are DNA barcodes. In particular embodiments, the spatial identifiers are disseminated through a tissue such that a gradient of spatial identifiers is created, allowing spatial reconstruction following disaggregation. Optionally, a single biological sample is subject to labeling with two or more different types of spatial identifiers such that overlapping gradients are created, and these multiple gradients are employed in determining spatial reconstruction of cells following disaggregation.

As indicated, following application of spatial identifiers to a tissue, the tissue can be disaggregated and separated into compartments. In particular embodiments, compartments contain a single cell. Compartments (or the cells within them) can be associated with a second label, referred to herein as a cellular label. The cellular label can include a genomic label and/or a transcriptional label. The genomic label allows identification of each compartment's cell following genomic analysis. The transcriptional label allows identification of each compartment's cell following transcriptional analysis. The spatial identifier and the compartment's or cell's cellular label can be computationally linked to provide information regarding the type of cell and its location within a tissue before tissue disaggregation.

### BRIEF DESCRIPTION OF THE FIGURES

FIGS. 1A-1G. FIG. 1A shows an arrangement of an identifiably labeled sequence in an oligonucleotide to barcode a molecular entity such as an antibody or a cDNA including a sequencing adapter (e.g., Illumina P7), an identifiably labeled sequence (e.g., a randomly generated barcode from 10x Genomics, Pleasanton, Calif.), a “read 2” sequence that provides a priming region for sequencing, a unique molecular identifier (UMI), and a poly dTVN. The poly dTVN sequence allows amplification of cDNA from the poly A tail end. ‘N’ is any base, ‘V’ is either A, C or G. This arrangement can be adapted to entities for delivery to the surface or interior of cells to be labeled. FIGS. 1B-1F show where the barcoded oligonucleotide integrates in the context of a single cell RNA sequencing (scRNA-seq) droplet-based platform (adapted from Zheng et al., Nat Commun. 8: 14049, 2017). (FIG. 1B) scRNA-seq workflow on GemCode™ technology platform. Cells are combined with reagents in one channel of a microfluidic chip, and gel beads from another channel to form Gel Beads in Emulsions (GEMs). Reverse transcription (RT) takes place inside each GEM, after which cDNAs are pooled for amplification and library construction in bulk. (FIG. 1C) Gel beads loaded with primers and barcoded oligonucleotides (transcriptional labels) are first mixed with cells and reagents, and subsequently mixed with oil-surfactant solution at a microfluidic junction. Single-cell GEMs are collected in the GEM outlet. (FIG. 1D) Percentage of GEMs containing 0 gel bead (N=0), 1 gel bead (N=1) and >1 gel bead (N>1). Data include five independent runs from multiple chip and gel bead lots over >70k GEMs for each run, n=5, mean±s.e.m. (FIG. 1E) Gel beads contain transcriptional labels including Illumina adapters, 10x barcodes, unique molecular identifiers (UMIs) and oligo dTs, which prime RT of polyadenylated RNAs. (FIG. 1F) Finished library molecules consist of Illumina adapters and sample indices, allowing pooling and sequencing of multiple libraries on a next-generation short read sequencer. (FIG. 1G) CellRanger pipeline workflow. Gene-barcode matrix (end box on right) is an output of the pipeline.

FIG. 2 shows an identifiably labeled antibody-oligonucleotide conjugate that recognizes a cell surface marker, and which can be used in the systems and methods of the present disclosure. An oligonucleotide can be attached to the antibody by a linker. The oligonucleotide includes a spatial identifier (illustrated as an antibody barcode) and can additionally have a PCR handle or strand switch sequence to allow amplification of the spatial identifier barcode for sequencing. The antibody can recognize a cell surface marker, for instance one that is specific for a cell type to be identified and/or localized, such as CD45 or keratin.

FIGS. 3A, 3B show exemplary options for delivering and/or positioning spatial identifiers to or within cells. FIG. 3A is a diagram of a magnetic nanoparticle that can be used to deliver spatial identifiers to cells. The spatial identifiers can include barcoded oligonucleotides with polyA tails. The spatial identifiers can be introduced into a cell, or can remain on the surface of a targeted cell, for instance by conjugation of the illustrated spatial identifier to an antibody or other targeting ligand that recognizes a marker on the target cell surface. FIG. 3B illustrates the ability of a magnetic field to align superparamagnetic nanoparticles; this can be used in the systems and methods of the present disclosure to direct spatial identifiers to regions of a tissue. Figure adapted from Bucak et al. Magnetic nanoparticles: synthesis, surface modifications and application in drug delivery. In Recent Advances in Novel Drug Carrier Systems 2012. InTech.

FIG. 4 shows an illustration of a magnetic nanoparticle that can be used in the systems and methods of the present disclosure. In the current disclosure, magnetic nanoparticles can be spatial identifiers and/or can be used to deliver spatial identifiers within a tissue. The spatial identifiers can include barcoded oligonucleotides with polyA tails. Figure adapted from Yezhelyev et al. (2006) The Lancet Oncology 7(8): 657-667.

FIG. 5 shows an exemplary option for delivering spatial identifier barcodes to cells. A tissue can be injected with spatial identifier barcodes by electroporation. The injection of spatial identifier barcodes can be temporally released and directionally controlled with an electric field. Figure adapted from Daniele Pugliesi at World Wide Web at commons.wikimedia.org/wiki/File:Moto_di_uno-ione_in_un_elettrolita.svq.

FIG. 6 shows a diagram of behaviors for various magnetic materials. Under the influence of a magnetic field, paramagnetic materials are magnetized, but when the magnetic field is removed this magnetization goes to zero. On the contrary, ferromagnetic materials present a remnant magnetization (MR) in the absence of the magnetic field. Superparamagnetic materials share properties of ferromagnetism and paramagnetism. When a spatial identifier barcode is delivered by magnetism, a number of parameters can be varied: the direction of delivery; the magnetic properties of the material used in delivery; the temporal release of spatial identifier barcodes; and the carrier nanomolecule. Figure adapted from World Wide Web at mappingignorance.org and physics.tutorvista.com.

FIG. 7, adapted from Dhavalikar & Rinaldi (J Magnetism Magnetic Mat 419: 267-273, 2016) shows additional methods to deliver spatial identifier barcodes into tissues to support spatial mapping. The asterisk (*) indicates an alternating magnetic field with time varying magnetic field independent of position. The triangle (▴) indicates a bias field with position varying magnetic field independent of time. The circle (●) indicates a saturated region where presence of a strong bias field aligns the particles in the direction of the bias field with the alternating magnetic field causing minor oscillations in the magnetization response. The square (▪) indicates a field free region where, due to absence of a bias field, the particles rotate freely with the alternating field to give a sinusoidal magnetization response.

FIGS. 8A-8C shows scatter plots and histograms of cells stained with fluorescently-labeled anti-CD45 and anti-CD102 antibodies prior to (pre-stained) or after (post-stained) dissociation of a tissue. (FIG. 8A) Scatter plots of cells pre-stained and post-stained. The percentages of viable cells are indicated. (FIG. 8B) Scatter plots and histogram of cells pre-stained and post-stained. Cells were labeled with a fluorescent anti-CD45 antibody. (FIG. 8C) Scatter plots and histogram of cells pre-stained and post-stained. Cells were labeled with a fluorescent anti-CD102 antibody.

### DETAILED DESCRIPTION

This disclosure provides systems and methods that allow identifying a cell's type and location in a biological sample following the biological sample's disaggregation. The systems and methods include labeling a biological sample (e.g., a tissue) having a three-dimensional structure and heterogenous cell-type composition with one or more spatial identifiers; disaggregating labeled cells from the tissue; analyzing the disaggregated labeled cells for the presence and/or quantity of the one or more spatial identifiers; and computationally mapping the cells into locations within a representation of the tissue sample based at least in part on the presence and/or quantity of the one or more spatial identifiers. The locations can be expressed or represented, e.g., in Cartesian, spherical, cylindrical, or other coordinate systems. Some examples include measuring portions of a labeled biological sample to associate coordinates with spatial-identifier distributions, then using data regarding those associations to determine coordinates for disaggregated cells based on their spatial-identifier distributions.

In specific embodiments, the current disclosure provides systems and methods that combine cellular spatial mapping with high-throughput single cell transcriptomics to correlate cell location with cell type within a biological structure. For example, in particular embodiments, the described systems and methods can be used to help unravel the biology of tumor-infiltrating immune cells and assess the clinical utility of characterizing these cells in relation to their position within a tumor. In particular embodiments, the systems and methods can be applied to comprehensively characterize the immune cell composition of non-small cell lung cancer, where the response rate to immune checkpoint inhibitor therapy is only 20%. This cancer highlights the current lack, and need for predictive factors of response to help guide future clinical trials and therapy.

There are many ways that the systems and methods of the present disclosure can be implemented. In general, implementation includes labeling a biological sample (such as a tissue sample having a three-dimensional structure and heterogenous cell-type composition) with one or more spatial identifiers; disaggregating labeled cells from the tissue; separating disaggregated cells into compartments; determining the cell type of compartmentalized cells, in particular embodiments through transcriptional analysis; analyzing the disaggregated labeled cells for the presence and/or quantity of the one or more spatial identifiers; computationally mapping the cells into locations within a representation of the tissue sample based at least in part on the presence and/or quantity of the one or more spatial identifiers; determining the cell type of the mapped cell.

The following provides an overview of one category of implementation, which uses barcode nucleic acid sequences as spatial identifiers, and includes the following aspects:

A biological sample to be analyzed is obtained.

Barcoded nucleic acid sequences are synthesized or obtained. The barcoded nucleic acids can optionally be conjugated to binding agents that recognize a cell surface marker on (or within) cells of the biological sample.

The barcoded nucleic acids are introduced into the biological sample to be analyzed. The introduction can include contacting a biological sample with binding agents conjugated to barcoded nucleic acids or with barcoded nucleic acids themselves. The binding agents conjugated to barcoded nucleic acids or barcoded nucleic acids can be delivered by, for example, diffusion, electroporation, magnetic charge motivation, particle bombardment, and/or pressure wave or ultrasound motivation. The binding agents conjugated to barcoded nucleic acids or barcoded nucleic acids can be delivered on a time delay. In particular embodiments, one type of known barcoded nucleic acid is introduced at one known location of the biological sample and another known, different barcoded nucleic acid is introduced at another known location of the biological sample. In particular embodiments, a plurality of known barcoded nucleic acids are introduced at a plurality of known locations of the biological sample, such that each known barcoded nucleic acid is introduced at one known location of the biological sample. This step generates spatial identifier barcodes disseminated throughout the biological sample in a manner that provides information to spatially re-create a cell's location following disaggregation. In some examples, at least a first one of the known locations is different from at least a second one of the known locations. For example, each of the known locations can be unique. Known locations can also be referred to as recorded locations.

The biological sample is then disaggregated and compartmentalized as single cells. A compartment (or partition) is associated with a cellular label that allows linking the single cell to a compartment or partition of origin. In particular embodiments, the cellular label includes a genomic label. In particular embodiments, the cellular label includes a transcriptional label.

Particular embodiments utilize sequencing to detect spatial identifier barcodes for the purpose of spatial mapping and engage in massively parallel digital transcriptional profiling of single cells for the purpose of identifying cell type associated with an identified location (see Zheng et al., Nature Communications, DOE: 10.1038/ncomms14049). As indicated, these embodiments can utilize systems and methods to partition individual cells into droplets or microdroplets, for instance as described in Zhang et al. (Scientific Reports 7: 41192, 2017), Terekhov et al. (PNAS USA, 114(10):2550-2555, 2017); Brouzes (Methods Mol Biol. 853:105-139, 2012); US20170260584. The following description provides detailed explanation and options to practice the systems and the methods of the current disclosure: (I) Biological Samples; (II) Spatial Identifiers; (Ill) Delivery of Spatial Identifiers to a Biological Sample; (IV) Imaging of a Biological Sample; (V) Disaggregation of a Labeled Biological Sample; (VI) Sequestration of Cells from a Labeled Biological Sample; (VII) Cellular Labels; (VIII) Computer Control Systems; (IX) Kits; (X) Exemplary Embodiments; and (XI) Examples.

(I) Biological Samples. As indicated, a first step involves obtaining a biological sample to be analyzed. In particular embodiments, a biological sample includes a tumor. The tumor can be derived from a living organism and/or can be a stored tumor sample. Biological samples are not limited to tumors, however, and include one or more cells, tissues, organs, or portions thereof. In particular, as used herein a biological sample for analysis is a sample containing two or more cells in a defined physical (e.g., 2-dimensional or 3-dimensional) relationship with each other.

In particular embodiments, a biological sample can be from a human being, a veterinary animal and/or a research animal. The sample can be derived from an organ, including for example, an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; an organ of the central nervous system such as brain, brainstem, cerebellum, spinal cord, cranial nerve, or spinal nerve; a sensory organ such as eye, ear, nose, or tongue; or an organ of the integument such as skin, subcutaneous tissue or mammary gland.

A biological sample can be considered (or suspected) healthy or diseased when used. In some cases, two samples can be used: a first being considered diseased and a second being considered as healthy (e.g. for use as a healthy control). Any of a variety of conditions can be evaluated, including an autoimmune disease, cancer, cystic fibrosis, aneuploidy, pathogenic infection, psychological condition, hepatitis, diabetes, sexually transmitted disease, heart disease, stroke, cardiovascular disease, multiple sclerosis or muscular dystrophy.

In particular embodiments, biological samples include at least one cell type to be targeted for analysis (e.g., TILs). In particular embodiments, biological samples include a cell type composition. Particular embodiments of a cell type composition include the number of a given cell type in a biological sample, a type of cell in a biological sample, or the number and type of a given cell type in a biological sample. A cell type can be characterized by many factors, including cell surface markers, morphology, size, shape, function, genomic sequences, and/or gene expression profile. In particular embodiments, a cell type composition can be uniform, where all cells are of the same type. In particular embodiments, a cell type composition can be heterogeneous, where at least one cell is of a different type than a plurality of other cells in the composition. In particular embodiments, biological samples include a rare cell type (for instance, where the cell type is less than 10% of the cells in the sample, less than 5% of the cells in the sample, less than 2% of the cells in the sample, less than 1% of the cells in the sample, or less than 0.5% of the cells in the sample) within a structurally heterogenous tissue sample.

In particular embodiments, biological samples, such as tumors, are obtained from humans or research animals, such as rats, mice, or non-human primates (NHP). In particular embodiments, biological samples can be obtained from any living organism that includes a 2-dimensionsal or three-dimensional cell structure, such as mammals, primates, humans, NHPs, rodents, mice, rats, rabbits, guinea pigs, ungulate, horse, sheep, pigs, goats, cows, cats, dogs; plants, Arabidopsis thaliana, corn, sorghum, oat, wheat, rice, canola, soybean; algae, Chlamydomonas reinhardtii, nematodes, Caenorhabditis elegance, insects, Drosophila melanogaster, mosquitos, fruit flies, honey bees, spiders, a fish such as zebrafish; a reptile; an amphibian such as a frog or Xenopus laevis; a Dictyostelium discoideum, fungi, Pneumocystis carinii, Takifugu rubripes, yeast, Saccharomyces cerevisiae, Schizosaccharomyces pombe, or Plasmodium falciparum. Samples can be derived from a homogeneous culture or population of the above organisms or alternatively from a collection of several different organisms, for example, in a community or ecosystem.

In particular embodiments, tissue clearing techniques are used to process biological samples. Techniques for tissue clearing are known in the art, for instance those discussed in Richardson & Lichtman (Cell 162(2):246-257, 2015), Azaripour et al. (Prog. Histochem Cytochem. 51(2):9-23, 2016), and Ariel (Int. J Biocem Cell Biol. 84:35-39, 2017).

(II) Spatial Identifiers. The systems and methods provided herein employ one or more spatial identifiers that are applied to a biological sample before tissue disaggregation. In this context, a spatial identifier is a molecule, particle, or other compound that is capable of being detected (directly or indirectly), distinguished from other spatial identifiers (particularly in embodiments in which more than one spatial identifier is used to characterize a single biological sample), capable of being delivered to and into the selected biological sample, and capable of producing a gradient of amount (or concentration) of the label across at least one axis of the biological sample to which it is applied. Within these limitations, the identifiable labels can be quite diverse in composition, form, and format. Representative example spatial identifiers (and classes of such labels) are described herein. It is particularly contemplated that embodiments of the systems and methods for mapping cells within a biological sample may employ spatial identifiers from more than one category—for instance, one or more labels that include a barcode nucleic acid used along with one or more labels detectable through detection of an electromagnetic frequency (such as for instance a fluorescent label or a radioactive label).

Exemplary types of spatial identifiers include barcoded nucleic acids (“spatial identifier nucleic acid barcode”), fluorescent molecules, radioactive molecules, chemiluminescent labels, spectral colorimetric labels, detectable tags, fluorescence emitting metals, and/or magnetic particles.

In some embodiments, systems and methods of the present disclosure use nucleic acids including identifiable (known) barcodes to assess where one or more cells are spatially in a biological sample. A barcode or barcode sequence can include a series of nucleotides in a nucleic acid that can be used to identify a cell or group of cells in a biological sample. In particular embodiments, a barcode sequence includes a random, unique sequence of nucleotides. In particular embodiments, the terms “spatial tag” or “spatial barcode” includes a nucleic acid having a sequence that is indicative of a location. Typically, the nucleic acid is a synthetic molecule having a sequence that is not found in one or more biological sample that will be used with the nucleic acid. However, in some embodiments the nucleic acid molecule can be naturally derived or the sequence of the nucleic acid can be naturally occurring, for example, in a biological sample that is used with the nucleic acid. The location indicated by a spatial tag can include a location in or on a biological sample. A barcode sequence can function as a spatial tag. A barcode sequence can be part of a nucleic acid sequence that contains other sequences, such as sequences for amplification of the nucleic acid, sequences useful in sequencing, and/or sequences useful for cDNA synthesis. In particular embodiments, one or more barcode sequences that are used with a biological sample are not present in the genome, transcriptome or other nucleic acids of the biological sample. For example, barcode sequences can have less than 80%, 70%, 60%, 50% or 40% sequence identity to any nucleic acid sequences in a particular biological sample.

Barcode sequences can be any of a variety of lengths. Longer sequences can generally accommodate a larger number and variety of barcodes. All barcoded nucleic acids in a plurality can have the same length barcode (albeit with different sequences), but it is also possible to use different length barcodes in different nucleic acids. A barcode sequence can be at least 2, 4, 6, 8, 10, 12, 15, 20 or more nucleotides in length. In particular embodiments, the length of the barcode sequence can be at most 20, 15, 12, 10, 8, 6, 4 or fewer nucleotides. In particular embodiments, a barcode sequence may have a length in range of from 4 to 36 nucleotides, or from 6 to 30 nucleotides, or from 8 to 20 nucleotides. Barcode sequences are described in, for example: U.S. Pat. No. 5,635,400; Brenner et al., Proc. Natl. Acad. Sci., 97:1665-1670, 2000; Shoemaker et al., Nature Genetics 14: 450-456, 1996; EP0799897; U.S. Pat. No. 5,981,179; US20140342921; and U.S. Pat. No. 8,460,865.

A nucleic acid including a barcode can additionally have one or more PCR handles for amplification of the nucleic acid. A PCR handle includes a universal or common sequence having a series of nucleotides that is common to two or more nucleic acid molecules even if the molecules also have regions of sequence that differ from each other. A universal sequence present in different members of a collection of molecules can allow the replication or amplification of multiple different nucleic acids using a population of universal primers that are complementary to the universal sequence. Thus, a universal primer includes a sequence that can hybridize specifically to a universal sequence. Barcoded nucleic acids can include a common sequence for amplification of the nucleic acids. Examples of PCR handles include 5′-TCGTCGGCAGCGTC (SEQ ID NO: 1, Illumina Nextera read1 handle sequence), 5′-GTCTCGTGGGCTCGG (SEQ ID NO: 2, Illumina Nextera read2 handle sequence), 5′-AATGATACGGCGACCACCGAGATCTACAC (SEQ ID NO: 3, Illumina handle), CAAGCAGAAGACGGCATACGAGAT (SEQ ID NO: 4, Illumina handle), and 5′-CCTTGGCACCCGAGAATTCC (SEQ ID NO: 5, Illumina TruSeq RNA read2 handle)

The term “primer” includes an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for some applications, depending on the complexity of the target sequence, the oligonucleotide primer may contain 15-25 or more nucleotides, although it may contain fewer nucleotides.

A nucleic acid including a barcode can include a unique molecular identifier (UMI). A UMI includes a random nucleotide sequence that can be used to establish a distinct identify for each input molecule that is sequenced. In particular embodiments, a UMI can include a random nucleotide sequence of 5-15 nucleotides, 7-13 nucleotides, or 8-12 nucleotides. In particular embodiments, a UMI can include a random nucleotide sequence of 10 nucleotides. In particular embodiments, molecules that share a UMI are derived from the same input molecule. In particular embodiments of using single cell RNA sequencing (scRNA-seq) to determine a cell type described herein, cDNA molecules sharing the same UMI can originate from the same mRNA molecule in a given cell. In particular embodiments, a UMI can reduce or eliminate effects of PCR amplification bias.

A nucleic acid including a barcode can additionally have strand switching sequences so that a copy of the barcoded nucleic acid can be generated.

The terms “polynucleotide,” “oligonucleotide,” “nucleic acid” and “nucleic acid molecule” are used interchangeably herein and include a polymeric form of nucleotides of any length, and may include ribonucleotides, deoxyribonucleotides, analogs thereof, or mixtures thereof. This term includes the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing non-nucleotidic backbones, for example, polyamide (e.g., peptide nucleic acids (“PNAs”)) and polymorpholino (commercially available from Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA:DNA hybrids can exhibit higher stability in some environments. The terms “polynucleotide,” “oligonucleotide,” “nucleic acid” and “nucleic acid molecule” can include any suitable length, such as at least 5, 6, 7, 8, 9, 10, 20, 30, 40, 50, 100, 200, 300, 400, 500, 1,000 or more nucleotides.

The terms “nucleoside” and “nucleotide” can include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” includes nucleosides and nucleotides.

The term “poly T or poly A,” when used in reference to a nucleic acid sequence, is intended to mean a series of two or more thiamine (T) or adenine (A) bases, respectively. A poly T or poly A can include at least 2, 5, 8, 10, 12, 15, 18, 20 or more of the T or A bases, respectively. In particular embodiments, a poly T or poly A can include at most 30, 20, 18, 15, 12, 10, 8, 5 or 2 of the T or A bases, respectively.

Fluorescent molecules that can be used as spatial identifiers include any molecule that when exposed to light of the proper wavelength, can then be detected due to fluorescence. Exemplary fluorescent molecules include acridine dyes; AMCA; Allophycocyanine; benzimide dyes; biotin; blue fluorescent proteins (e.g. eBFP, eBFP2, Azurite, mKalamal, GFPuv, Sapphire, T-sapphire); BODIPY dyes; carbazole dyes; 6-carboxy-4′,5′-dichloro-2′,7′-dimethoxy fiuorescein (JOE or J); 6-carboxyfhiorescein (commonly known by the abbreviations FAM and F); 6-carboxy-2′,4′,7′,4,7-hexachlorofiuorescein (HEX); 5-carboxyrhodamine-6G (R6G5 or G5); 6-carboxy-X-rhodamine (ROX or R); 6-carboxyrhodamine-6G (R6G6 or G6); Coumarins; cyan fluorescent proteins (e.g. eCFP, Cerulean, CyPet, AmCyanl, Midoriishi-Cyan); Cyanine; cyanine dyes (e.g. Cy3, Cy5 and Cy7); ethidium dyes; fluorescein; fluorescamine; fluorescein isothiocyanate (FITC); green fluorescent proteins (e.g. GFP, GFP-2, tagGFP, turboGFP, eGFP, Emerald, Azami Green, Monomeric Azami Green, CopGFP, AceGFP, ZsGreenI, Oregon Green™ (Thermo Fisher Scientific)); Hoechst 33258; Luciferase; orange fluorescent proteins (mOrange, mKO, Kusabira-Orange, Monomeric Kusabira-Orange, mTangerine, tdTomato); peridenin chlorophyll; phenanthridine dyes; phenoxazine dyes; phycoerythrin; phycocyanin; polymethine dyes; porphyrin dyes; N,N,N′,N′-tetramethyl-6carboxyrhodamine (TAMRA or T); o-phthaldehyde; quinoline dyes; red fluorescent proteins (mKate, mKate2, mPlum, DsRed monomer, mCherry, mRFP1, DsRed-Express, DsRed2, DsRed-Monomer, HcRed-Tandem, HcRedI, AsRed2, eqFP611, mRaspberry, mStrawberry, Jred, Texas Red™ (Thermo Fisher Scientific)); rhodamine; rhodamine 110; tetrarhodimine isothiocynate (TRITC)); umbelliferone; yellow fluorescent proteins (e.g., YFP, eYFP, Citrine, Venus, YPet, PhiYFP, ZsYellowI); and tandem conjugates such as phycoerythrin-cyanine.

In some embodiments, a spatial identifier includes a chemiluminescent label, including, for example, lucigenin, luminol, luciferin, isoluminol, theromatic acridinium ester, imidazole, acridinium salt, or oxalate ester.

In some embodiments, a spatial identifier can include a spectral colorimetric label including colloidal gold or colored glass or plastic (e.g., polystyrene, polypropylene, and latex) beads.

In some embodiments, spatial identifiers can include a detectable tag. Exemplary tags include STREPTAG® (GmbH, LLC, Gottingen, Del.), STREP® tag II (WSHPQFEK (SEQ ID NO: 6)), or any variant thereof; see, e.g., U.S. Pat. No. 7,981,632), His tag, Flag tag (DYKDDDDK (SEQ ID NO:7)), Xpress tag (DLYDDDDK (SEQ ID NO: 8)), Avi tag (GLNDIFEAQKIEWHE (SEQ ID NO: 9)), Calmodulin tag (KRRWKKNFIAVSAANRFKKISSSGAL (SEQ ID NO: 10)), Polyglutamate tag, HA tag (YPYDVPDYA (SEQ ID NO: 11)), Myc tag (EQKLISEEDL (SEQ ID NO: 12)), Nus tag, S tag, SBP tag, Softag 1 (SLAELLNAGLGGS (SEQ ID NO:13)), Softag 3 (TQDPSRVG (SEQ ID NO: 14)), and V5 tag (GKPIPNPLLGLDST (SEQ ID NO: 15)).

A spatial identifier can also be detectably labeled using fluorescence emitting metals such as 152Eu, or others of the lanthanide series. These metals can be attached to the reagent using such metal chelating groups as diethylenetriaminepentaacetic acid (DTPA) or ethylenediaminetetraacetic acid (EDTA).

In some embodiments, a spatial identifier can include an enzyme including horseradish peroxidase and alkaline phosphatase. These spatial identifier enzymatic labels can produce, for example, a chemiluminescent signal, a color signal, or a fluorescent signal. Enzymes contemplated for use to spatially identify cells in a biological sample include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In some embodiments, a spatial identifier can include a radiolabeled molecule (e.g., includes a radioactive isotope). Examples of radioactive isotopes include ²²⁸AG, ²²⁸AG, ²²⁸AG, ¹⁰⁵Ag, ¹⁰⁶mAg, ¹¹⁰mAg, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As, ⁷⁶As, ⁷⁷As, 209At, 210At, 191Au, 192Au, 193Au, 194Au, 195Au, ¹⁹⁶Au, ¹⁹⁶m²Au, ¹⁹⁸Au, ¹⁹⁸mAu, ¹⁹⁹Au, ²⁰⁰mAu, ¹²⁸Ba, ¹³¹Ba, ¹³³mBa, ¹³⁵mBa, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk, ²⁴⁵Bk, ²⁴⁶Bk, ²⁴⁸mBk, ²⁵⁰Bk, ⁷⁶Br, ⁷⁷Br, ⁸⁰mBr, ⁸²Br, ¹¹C, ¹⁴C, ⁴⁵Ca, ⁴⁷Ca, ¹⁰⁷Cd, ¹¹⁵Cd, ¹¹⁵mCd, ¹¹⁷mCd, ¹³²Ce, ¹³³mCe, ¹³⁴Ce, ¹³⁵Ce, ¹³⁷Ce, ¹³⁷mCe, ¹³⁹Ce, ¹⁴¹Ce, ¹⁴³Ce, ¹⁴⁴Ce, ²⁴⁶Cf, ²⁴⁷Cf, ²⁵³Cf, ²⁵⁴Cf, ²⁴⁰ Cm, ²⁴¹Cm, ²⁴²Cm, ²⁵²Cm, ⁵⁵CO, ⁵⁶CO, ⁵⁷CO, ⁵⁸CO, ⁵⁸mCO, ⁶⁰CO, ⁴⁸Cr, ⁵¹Cr, ¹²⁷Cs, ¹²⁹Cs, ¹³¹Cs, ¹³²Cs, ¹³⁶Cs, ¹³⁷Cs, ⁶¹Cu, ⁶²Cu,⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er, ¹⁶¹Er, ¹⁶⁵Er, ¹⁶⁹Er, ¹⁷¹Er, ¹⁷²Er, ²⁵° Es, ²⁵¹Es, ²⁵³ES, ²⁵⁴ES, ²⁵⁴mES, ²⁵⁵ES, ²⁵⁶mES, ¹⁴⁵Eu, ¹⁴⁶Eu, ¹⁴⁷Eu, ¹⁴⁸Eu, ¹⁴⁹Eu, ¹⁵⁰mEu, ¹⁵²mEu, ¹⁵⁶Eu, ¹⁵⁷Eu, ⁵²Fe, ⁵⁹Fe, ²⁵¹Fm, ²⁵²Fm, ²⁵³Fm, ²⁵⁴Fm, ²⁵⁵Fm, ²⁵⁷Fm, ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge, ¹⁷⁰Hf, ¹⁷¹Hf, ¹⁷³Hf, ¹⁷⁵Hf, ¹⁷⁹m²Hf, ¹⁸⁰mHf, ¹⁸¹Hf, ¹⁸⁴Hf, ¹⁹²Hg, ¹⁹³Hg, ¹⁹³mHg, ¹⁹⁵Hg, ¹⁹⁵mHg, ¹⁹⁷Hg, ¹⁹⁷mHg, ²⁰³Hg, ¹⁶⁰mHo, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰In, ¹¹¹In, ¹¹⁴mIn, ¹¹⁵mIn, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ¹⁹⁰m²Ir, ¹⁹²Ir, ¹⁹³mIr, ¹⁹⁴Ir, ¹⁹⁴m²Ir, ¹⁹⁵mIr, ⁴²K, ⁴³K, ⁷⁶Kr, ⁷⁹Kr, ⁸¹mKr, ⁸⁵mKr, ¹³²La, ¹³³La, ¹³⁵La, ¹⁴⁰La, ¹⁴¹ La, ²⁶²Lr, ¹⁶⁹Lu, ¹⁷⁰Lu, ¹⁷¹Lu, ¹⁷²Lu, ¹⁷⁴mLu, ¹⁷⁶mLu, ¹⁷⁷Lu, ¹⁷⁷mLu, ¹⁷⁹Lu, ²⁵⁷Md, ²⁵⁸Md, ²⁶⁰Md, ²⁸Mg, ⁵²Mn, ⁹⁰Mo, ⁹³mMo, ⁹⁹Mo, ¹³N, ²⁴Na, ⁹⁰Nb, ⁹¹mNb, ⁹²mNb, ⁹⁵Nb, ⁹⁵mNb, ⁹⁶Nb, ¹³⁸Nd, ¹³⁹mNd, ¹⁴⁰Nd, ¹⁴⁷Nd, ⁵⁶Ni, ⁵⁷Ni, ⁶⁶Ni, ²³⁴Np, ²³⁶mNp, ²³⁸Np, ²³⁹Np, ¹⁵O, ¹⁸²Os, ¹⁸³Os, ¹⁸³mOs, ¹⁸⁵Os, ¹⁸⁹mOs, ¹⁹¹Os, ¹⁹¹mOs, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd, ¹¹¹mPd, ¹¹²Pd, ¹⁴³Pm, ¹⁴⁸Pm, ¹⁴⁸mPm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁸⁹Pt, ¹⁹¹Pt, ¹⁹³mPt, ¹⁹⁵mPt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu, ²²³Ra, ²²⁴Ra, ²²⁵Ra, ⁸¹ Rb, ⁸²Rb, ⁸²mRb, ⁸³Rb, ⁸⁴Rb, ⁸⁶Rb, ¹⁸¹Re, ¹⁸²Re, ¹⁸²mRe, ¹⁸³Re, ¹⁸⁴Re, ¹⁸⁴mRe, ¹⁸⁶Re, ¹⁸⁸Re, ¹⁸⁹Re, ¹⁹⁰mRe, ⁹⁹Rh, ⁹⁹mRh, ¹⁰⁰Rh, ¹⁰¹mRh, ¹⁰²Rh, ¹⁰³mRh, ¹⁰⁵Rh, ²¹¹Rh, ²²²Rn, ⁹⁷Ru, ¹⁰³Ru, ¹⁰⁵Ru, ³⁵S, ¹¹⁸mSb, ¹¹⁹Sb, ¹²⁰Sb, ¹²⁰mSb, ¹²²Sb, ¹²⁴Sb, ¹²⁶Sb, ¹²⁷Sb, ¹²⁸Sb, ¹²⁹Sb, ⁴³Sc, ⁴⁴Sc, ⁴⁴mSc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ¹¹⁷mSn, ¹¹⁹mSn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta, ¹⁸³Ta, ¹⁸⁴Ta, ¹⁴⁹Tb, ¹⁵⁰Tb, ¹⁵¹Tb, ¹⁵²Tb, ¹⁵³Tb, ¹⁵⁴Tb, ¹⁵⁴mTb, ¹⁵⁴m²Tb, ¹⁵⁵Tb, ¹⁵⁶Tb, ¹⁵⁶mTb, ¹⁵⁶m²Tb, ¹⁶⁰Tb, ¹⁶¹Tb, ⁹⁴Tc, ⁹⁵Tc, ⁹⁵mTc, ⁹⁶Tc, ⁹⁷mTc, ⁹⁹mTc, ¹¹⁸Te, ¹¹⁹Te, ¹¹⁹mTe, ¹²¹Te, ¹²¹mTe, ¹²³mTe, ¹²⁵mTe, ¹²⁷Te, ¹²⁷mTe, ¹²⁹mTe,¹³¹mTe, ¹³²Te, ²²⁷Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ¹²⁹mXe, ¹³¹mXe, ¹³³Xe, ¹³³mXe, ¹³⁵Xe, ⁸⁵mY, ⁸⁶Y, ⁸⁷Y, ⁸⁷mY, ⁸⁸Y, ⁹⁰Y, ⁹⁰mY, ⁹¹Y, ⁹²Y, ⁹³Y, ¹⁶⁶Yb, ¹⁶⁹Yb, ¹⁷⁵Yb, ⁶²Zn, ⁶⁵Zn, ⁶⁹mZn, ⁷¹mZn, ⁷²Zn, ⁸⁶Zr, ⁸⁸Zr, ⁸⁹Zr, ⁹⁵Zr, and ⁹⁷Zr.

Methods for preparing radioimmunoconjugates are established in the art. Examples of radioimmunoconjugates are commercially available, including Zevalin™ (DEC Pharmaceuticals) and Bexxar™ (Corixa Pharmaceuticals), and similar methods can be used to prepare radio-conjugates to be used in the methods and systems described herein.

Magnetic particles can serve multiple roles in the context of the systems and methods provided herein. For instance, magnetic particles may themselves be considered spatial identifiers in that magnetic particles can be detected in or on cells after delivery thereto. Where magnetic particle(s) are used as the spatial identifier, there are contemplated systems in which different size particles or different strengths of magnetism are used as differential labels that can be distinguished from each other after delivery to the biological sample. Magnetic nanoparticles are described for instance in Mohammed et al., Particulogy 30(2017):1-14, and references cited and reviewed therein.

In particular embodiments, spatial identifiers can include or be associated with targeting/binding agents that direct (target) the spatial identifier to a specific cell or cell type, sequences for amplifying a detectable signal from the spatial identifier (such as nucleic acid sequences useful in enabling copying of a sequence of interest before it is sequenced, or photomultiplier compounds that increase the detectability of EM frequency(s)), or other additional elements. Some spatial identifiers may be associated with more than one additional, optional component.

Particular embodiments include binding agents conjugated to a spatial identifier. A binding agent includes a molecule that can bind a ligand on the surface of one or more cells of a biological sample. In particular embodiments, the binding agent includes an antibody or functional fragment thereof. The terms “antibody” and “immunoglobulin” are used interchangeably herein and are well understood by those in the field. Those terms refer to a protein including one or more polypeptides that specifically binds an antigen. One form of antibody includes the basic structural unit of an antibody. This form is a tetramer and includes two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of 25 kDa or 214 amino acids) include a variable region of 110 amino acids at the NH₂-terminus and a kappa or lambda constant region at the COOH-terminus. Full-length immunoglobulin “heavy chains” (of 50 kDa or 446 amino acids), similarly include a variable region (of 116 amino acids) and one of the aforementioned heavy chain constant regions, e.g., gamma (of 330 amino acids).

Particular embodiments of antibodies and immunoglobulins include antibodies or immunoglobulins of any isotype, fragments of antibodies which retain specific binding to an antigen, including, for example, Fab, Fv, scFv, and Fd fragments; chimeric antibodies; humanized antibodies; single-chain antibodies; and fusion proteins including an antigen-binding portion of an antibody and a non-antibody protein. The antibodies may be detectably labeled, e.g., with a radioisotope, an enzyme which generates a detectable product, a fluorescent protein, a fluorescent molecule, or a stable elemental isotope. The antibodies may be further conjugated to other moieties, such as members of specific binding pairs, e.g., biotin (member of a biotin-avidin specific binding pair), and the like.

Antibodies may exist in a variety of other forms including, for example, bi-functional (i.e. bi-specific) hybrid antibodies (e.g., Lanzavecchia et al. (1987) Eur. J. Immunol. 17: 105) and in single chains (e.g., Huston et al. (1988) Proc. Natl. Acad. Sci USA 85: 5879-5883 and Bird et al. (1988) Science 242: 423-426. See, generally, Hood et al. (1984) “Immunology”, N.Y., 2nd ed., and Hunkapiller and Hood (1986) Nature 323: 15-16).

An immunoglobulin light or heavy chain variable region consists of a “framework” region (FR) interrupted by three hypervariable regions, also called “complementarity determining regions” or “CDRs”. The extent of the framework region and CDRs has been precisely defined (see, “Sequences of Proteins of Immunological Interest” E. Kabat et al. (1991) US Department of Health and Human Services). In particular embodiments, the numbering of an antibody amino acid sequence can conform to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Chimeric antibodies are antibodies whose light and heavy chain genes have been constructed, typically by genetic engineering, from antibody variable and constant region genes belonging to different species. For example, the variable segments of the genes from a rabbit monoclonal antibody may be joined to human constant segments, such as gamma 1 and gamma 3.

The term “attached” can refer to the state of two things being joined, fastened, adhered, connected or bound to each other. For example, a spatial identifier such as a barcoded nucleic acid can be attached to a binding agent by a covalent or non-covalent bond. A covalent bond is characterized by the sharing of pairs of electrons between atoms. Exemplary covalent linkages include, for example, those that result from the use of click chemistry techniques and disulfide bonds resulting from oxidation of two -SH groups. A non-covalent bond is a chemical bond that does not involve the sharing of pairs of electrons and can include, for example, hydrogen bonds, ionic bonds, van der Waals forces, hydrophilic interactions and hydrophobic interactions. Exemplary non-covalent linkages can also include affinity interactions, receptor-ligand interactions, antibody-epitope interactions, avidin-biotin interactions, streptavidin-biotin interactions, and lectin-carbohydrate interactions.

Linkers can be used to conjugate a spatial identifier to a binding agent. Linkers are commonly known in the art and include those described in, for example, Denardo et al., Clin Cancer Res. 4(10):2483-2490, 1998; Peterson et al., Bioconjug. Chem. 10(4):553-557, 1999; and Zimmerman et al., Nucl. Med. Biol. 26(8):943-50, 1999. In particular embodiments, the linker can be a polyA sequence or a different oligo sequence. In particular embodiments, antibody-oligo conjugates can include those used in CITE-seq, i.e. with a polyA tail at the 3′ end leading to amplification of the antibody-derived barcode when cDNA is generated. Exemplary linkages are also descried in U.S. Pat. Nos. 6,737,236; 7,259,258; 7,375,234; 7,427,678; and US20110059865.

Particular embodiments of the systems and methods disclosed herein use cell surface markers as targets or ligands for the binding agents disclosed herein to direct the association of spatial identifiers with cells. In particular embodiments, cell surface markers that can be bound by a binding agent include: CD1a, CD1b, CD1c, CD2, CD3CD3E, CD4, CDS, CD7, CD8, CD9, CD10, CD11a, CD11b, CD11c, CD14, CD16, CD19, CD20, CD21, CD22, CD23, CD25, CD26, CD27, CD28, CD29, CD23, CD31, CD32, CD33, CD34, CD38, CD40, CD43, CD44, CD45, CD45R, CD45RA, CD49b, CD49d, CD49e, CD51, CD54, CD56, CD57, CD59, CD62E, CD64, CD68, CD69, CD71, CD80, CD83, CD86, CD90, CD93, CD94, CD105, CD107a, CD110, CD111, CD117/c-Kit, CD119, CD123, CD135, CD144, CD146, CD150, CD161, CD183, CD184 (CXCR4), CD195, CD202b, CD203c, CD208, CD209, CD227, CD243, CD271, CD278/ICOS, CD298, CD338, T cell receptor, FcγRII, FcεRI, high affinity IgE receptor, IFNαR/TNFR-1, CXC chemokine receptor, CCR1, CCRS, CCR7, Daffy antigen receptor for chemokines (DARC), natural cytotoxicity receptor, PDGF receptor, FGFR3, VEGFR1, VEGFR2, VEGFR3, TLR7, TLR9, IL-1R, IL-18R, LT-betaR, Tim-3, MHC class I, MHC class II, HLA-DR, glycophorin A, keratin, E-cadherin, integrin, B2M. In particular embodiments, cell surface markers that can be bound by a binding agent include: CD3, CD4, and/or CD8. In particular embodiments, the binding agents that bind CD3, CD4, and/or CD8 include an anti-CD3 antibody, an anti-CD4 antibody, and/or an anti-CD8 antibody, respectively. In particular embodiments, the binding agents that bind CD45 include an anti-CD45 antibody. In particular embodiments, the binding agents that bind CD45 include an anti-CD45 antibody conjugated with a fluorophore. Anti-CD45 antibodies are commercially available and include: mouse anti-human CD45 IgG1 antibody (Cat No. MBS396561, MyBioSource, Inc., San Diego, Calif.); rabbit polyclonal anti-CD45 (Cat No. GTX116018, GeneTex, Inc., Irvine, Calif.); and fluorescein mouse monoclonal H130 IgG1 kappa (Cat No. 200-302-N68, Rockland antibodies & assays, Limerick, Pa.). In particular embodiments, the binding agents that bind CD102 include an anti-CD102 antibody. In particular embodiments, the binding agents that bind CD102 include an anti-CD102 antibody conjugated with a fluorophore. Anti-CD102 antibodies are commercially available and include Alexa Fluor® 647 anti-mouse CD102 antibody clone 3C4 (MIC2/4) (Cat No. 105611, BioLegend, San Diego, Calif.); mouse anti-human CD102 IgG1 antibody (Cat No. MBS212037, MyBioSource, Inc., San Diego, Calif.); and rabbit polyclonal anti-CD102 (Cat No. NBP2-16912, Novus Biologicals, LLC, ntennial, Colo.).

In particular embodiments, cell surface markers for the binding agents to direct the association of spatial identifiers with cells include Prostate-Specific Membrane Antigen (PSMA), Wilms' Tumor 1 Antigen (WT1), Prostate Stem Cell Antigen (PSCA), Simian Vacuolating Virus 40 large T antigen (SV40 T), human epidermal growth factor 2 (HER2), receptor tyrosine kinase-like orphan receptor (ROR1), L1 cell adhesion molecule (L1-CAM), extracellular domain of MUC16 (MUC-CD), folate binding protein (folate receptor), Lewis Y carbohydrate antigen, or carboxy-anhydrase-IX (CAIX). In particular embodiments, the cell surface markers are expressed on cancer cells.

In particular embodiments, the binding agent includes an anti-CD44 antibody or binding fragment thereof, an anti-PSMA antibody or binding fragment thereof, an anti-WT1 antibody or binding fragment thereof, an anti-PSCA antibody or binding fragment thereof, an anti-SV40 T antibody or binding fragment thereof, an anti-HER2 antibody or binding fragment thereof, an anti-ROR1 antibody or binding fragment thereof, an anti-L1-CAM antibody or binding fragment thereof, an extracellular domain of MUC-CD antibody or binding fragment thereof, an anti-folate receptor antibody or binding fragment thereof, an anti-Lewis Y antibody or binding fragment thereof, an anti-mesothelin antibody or binding fragment thereof, or an anti-CAIX antibody or binding fragment thereof.

Flow data can show that spatial identifiers including cell-type-identifying antibodies and DNA oligos/barcodes penetrate into biological samples and remain tightly bound to target cells even after tissue disaggregation. As a result, each cell is associated with a presence, level, and/or amount of one or more spatial identifier, and the presence, level, and/or amount of the one or more spatial identifiers associated with each cell can help in determining the location of the cell within a biological sample. In particular embodiments, the amount or level of a spatial identifier can include a ratio of 2 spatial identifiers, 3 spatial identifiers, 4 spatial identifiers, 5 spatial identifiers, or more.

In particular embodiments, the spatial identifiers can be delivered directly to the biological sample, without the need for additional component(s) to be included for instance to serve as a delivery vehicle for the spatial identifier molecule/moiety itself. Such delivery can be considered naked label delivery, where spatial identifiers are provided directly to the biological sample without being attached or associated with other active components. Such direct, naked delivery may be carried out for instance using nucleic acids (either DNA or RNA, or derivatives thereof).

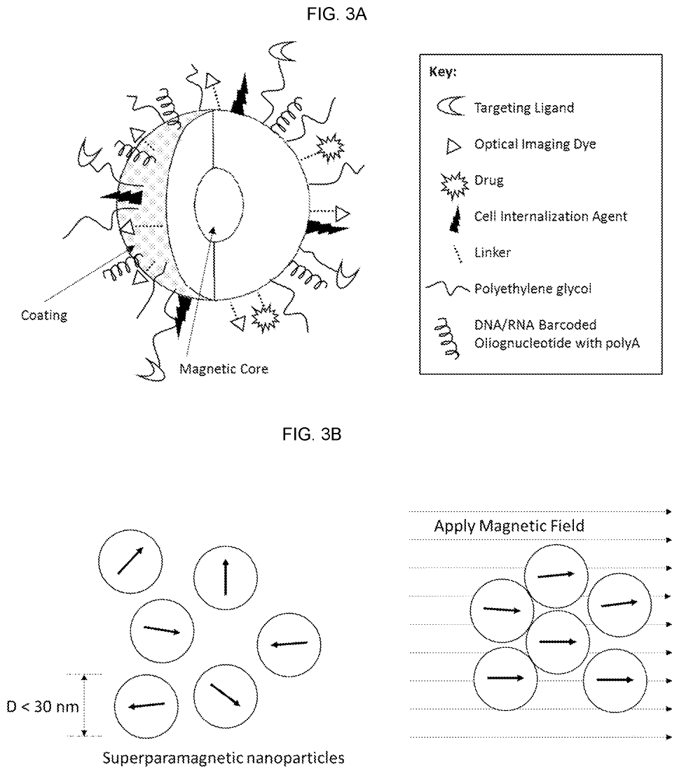

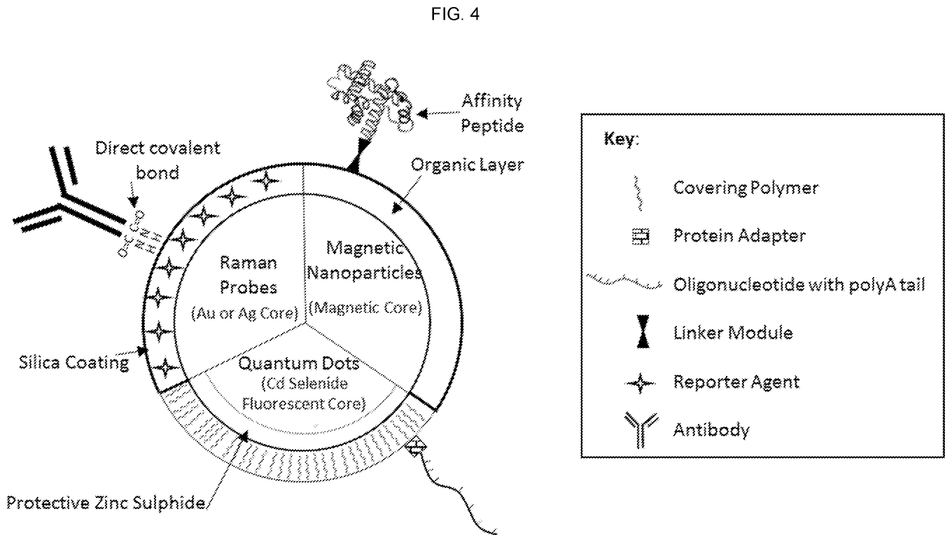

In particular embodiments, spatial identifiers are delivered in association with (for instance, covalently or otherwise bound to) a structural support carrier such as a nanoparticle, a magnetic particle, a polymer, a quantum dot, a raman probe, and so forth. FIGS. 3A, 3B and 4 provide schematic information on representative particle types that may be used in embodiments provided herein.

By way of example, the following publications describe the construction and/or use of one or more types of nanoparticle carriers that are useful in various embodiments of the provided systems and methods: Mohammed et al. (“Magnetic nanoparticles for environmental and biomedical applications: A review” Particuology 30:1-14, 2017); Williams et al. (“Magnetic Nanoparticle Drug Carriers and their Study by Quadrupole Magnetic Field-Flow Fractionation” Mol Pharm 6(5):1290-1306, 2009); Wang & Cuschieri (“Tumour Cell Labelling by Magnetic Nanoparticles with Determination of Intracellular Iron Content and Spatial Distribution of the Intracellular Iron” Int J Mol Sci. 14:9111-9125, 2013); Neurberger et al. (“Superparamagnetic nanoparticles for biomedical applications: Possibilities and limitations of a new drug delivery system” J Magnetism Magnetic Mat. 293:483-496, 2005); Hofmann-Amtenbrink et al. (Superparamagnetic nanoparticls for biomedical applications” Nanostructured Materials for Biomedical Applications, Chapter 12, 2009; ISBN: 978-81-7895-397-7); Ghazanfari et al. (“Perspective of Fe3O4 Nanoparticles Role in Biomedical Applications” Biochem Res Int. Article #7840161 (32 pages), 2016); Gao et al. (“Intracelluar Spatial Control of Fluorescent Magnetic Nanoparticles” JACS Comm. 130:3710-3711, 2008); Oude Engberink et al. (“Magnetic Resonance Imaging of Monocytes Labeled with Ultrasmall Superparamagnetic Particles of Iron Oxide Using Magnetoelectroporation in an Animal Model of Multiple Sclerosis” Mol. Imaging 9(5):268-277, 2010); Plank et al. (“Magnetically enhanced nucleic acid delivery. Ten years of magnetofection—Progress and prospects” Adv Drug Delivery Rev. 63:1300-1331, 2011); Patitsa et al. (“Magnetic nanoparticles coated with polyarabic acid demonstrate enhanced drug delivery and imaging properties for cancer theranostic applications” Scientific Reports 7:775 (8 pages), 2017; doi: 10.1038/s41598-017-00836-7); Mody et al. (“Magnetic nanoparticle drug delivery systems for targeting tumor” Appl. Nanosci 4:385-392, 2014); and Ulbrich et al. (“Targeted Drug Delivery with Polymers and Magnetic Nanoparticles: Covalent and Noncovalent Approaches, Release Control, and Clinical Studies” Chem Rev 116:5338-5431, 2016).

(III) Delivery of Spatial Identifiers to a Biological Sample. Integral to the systems and methods described herein is delivery of one or more spatial identifiers to the biological sample, thereby creating one or more gradients of spatial identifiers across a biological sample. These gradient(s) allow/enable spatial mapping of two or more cells from the biological sample. The mode of delivery of spatial identifiers in some instances will be influenced by the type of label(s) being used, the number of spatial identifiers being applied, the type of biological sample being analyzed (including the source of the sample, its form or shape, and so forth), and other variables.

Though specific methods of spatial identifier delivery are contemplated (including acoustical methods, electrophoresis, electroporation, heat assisted delivery, iontophoresis, light or another electromagnetic mobilization, magnetophoresis, microneedles, nanoporation, needle-free injection, piezoelectric droplet jet dispersion, a pump short-duration shock wave, sonophoresis, thermal droplet jet dispersion, ultrasound, low-frequency ultrasound, diffusion, biolistic (particle) bombardment, etc.) and some are discussed in detail in the following paragraphs, it is worthwhile first to discuss some attributes that may generally apply to all modes of delivery.

Delivery of a spatial identifier is made toward (or at) a particular side or location on or in the biological sample, and in particular embodiments, the point (or area, or region) of initial delivery is noted and used as a reference point in determining the map (spatial) position of one or more cells from the biological sample. In embodiments where more than one spatial identifier is applied to the same biological sample, the areas to which the spatial identifiers are applied can be selected to be different for different labels, or optionally for each spatial identifier that is applied, so that the spatial identifier gradient for each spatial identifier is different through the biological sample. Deconvolution of the differential gradient of spatial identifier(s) permits pinpoint mapping of individual cells (after disaggregation) to their location in the original biological sample that was labeled with spatial identifiers. Alternatively, differential spatial identifier gradients can be produced by applying different types of labels, or by applying different (or differential) motive force(s) to move (impel) the label into or through the biological sample. Thus, differential labeling may be accomplished even in instances where two or more labels are applied to the biological sample from substantially the same initial application area.

In some embodiments, spatial identifiers are permitted to diffuse across/into the biological sample from the point (or area, or region) of initial delivery. The rate of diffusion is influenced by the size and shape of the spatial identifier composition, its charge, and characteristics of the biological sample being analyzed. Diffusion produces a gradient of spatial identifiers that is higher (more dense, more concentrated) closer to the initial delivery area and lower (less dense, less concentrated) in cells that are further away from the initial delivery area.

In particular embodiments, spatial identifiers are delivered to a biological sample using electroporation—that is, the process of delivering a molecule into cells using pulse(s) of electricity that open (temporary) pores or channels in the cellular membranes. Techniques and technologies for electroporation of different cell types, including cells in tissues, are well known in the art. See, for instance, Potter & Heller (“Transfection by Electroporation” Curr Protoc Mol Biol. 9.3, 2003; doi: 10.1002/0471142727.mb0903s62) Chen et al. (“Membrane electroporation theories: a review”, Med Biol Engin Comput. 44(1-2):5-14, 2006), Yarmush et al. (“Electroporation-based Technologies for Medicine: Principles, Applications, and Challenges”, Ann. Rev. Biomed. Engin. 16:295-320, 2014; Kim & Lee (“Electroporation for nanomedicine: a review”, J Materials Chem B 5:2726-2738, 2017).

Electroporation environment and external factors may influence the effectiveness of spatial identifier delivery using electroporation. Depending on the embodiment, relevant variables may include temperature (particularly since electroporation may cause a significant increase in sample temperature), concentration of the spatial identifier being delivered, buffer conditions under which the electroporation is carried out (at least because the pH and buffer capacity of the electroporation may influence effectiveness), and so forth. Such variables can be determined and influenced by one of skill in the art to fit the specific sample being analyzed, as well as the spatial identifier(s) being applied.

It is contemplated in some instances that electroporation alone may be the delivery method for some spatial identifiers. In other embodiments, however, electroporation may be coupled with another delivery method (such as diffusion, magnetic motivation, or biolistic particle bombardment) to increase the uptake of spatial identifiers by cells once the spatial identifiers have been introduced into the biological sample.

In particular embodiments, delivery of spatial identifiers can also be facilitated through use of magnetic (nano)particles and magnetic field motivation. Methods of using magnetic (nano)particles for delivery of substances to biological samples are known; see, for instance, Mohammed et al. (“Magnetic nanoparticles for environmental and biomedical applications: A review” Particuology 30:1-14, 2017); Williams et al. (“Magnetic Nanoparticle Drug Carriers and their Study by Quadrupole Magnetic Field-Flow Fractionation” Mol Pharm 6(5):1290-1306, 2009); Wang & Cuschieri (“Tumour Cell Labelling by Magnetic Nanoparticles with Determination of Intracellular Iron Content and Spatial Distribution of the Intracellular Iron” Int J Mol Sci. 14:9111-9125, 2013); Neurberger et al. (“Superparamagnetic nanoparticles for biomedical applications: Possibilities and limitations of a new drug delivery system” J Magnetism Magnetic Mat. 293:483-496, 2005); Hofmann-Amtenbrink et al. (Superparamagnetic nanoparticls for biomedical applications” Nanostructured Materials for Biomedical Applications, Chapter 12, 2009; ISBN: 978-81-7895-397-7); Ghazanfari et al. (“Perspective of Fe3O4 Nanoparticles Role in Biomedical Applications” Biochem Res Int. Article #7840161 (32 pages), 2016); Gao et al. (“Intracelluar Spatial Control of Fluorescent Magnetic Nanoparticles” JACS Comm. 130:3710-3711, 2008); Oude Engberink et al. (“Magnetic Resonance Imaging of Monocytes Labeled with Ultrasmall Superparamagnetic Particles of Iron Oxide Using Magnetoelectroporation in an Animal Model of Multiple Sclerosis” Mol. Imaging 9(5):268-277, 2010); Plank et al. (“Magnetically enhanced nucleic acid delivery. Ten years of magnetofection—Progress and prospects” Adv Drug Delivery Rev. 63:1300-1331, 2011); Patitsa et al. (“Magnetic nanoparticles coated with polyarabic acid demonstrate enhanced drug delivery and imaging properties for cancer theranostic applications” Scientific Reports 7:775 (8 pages), 2017; doi: 10.1038/s41598-017-00836-7); Mody et al. (“Magnetic nanoparticle drug delivery systems for targeting tumor” Appl. Nanosci 4:385-392, 2014); and Ulbrich et al. (“Targeted Drug Delivery with Polymers and Magnetic Nanoparticles: Covalent and Noncovalent Approaches, Release Control, and Clinical Studies” Chem Rev 116:5338-5431, 2016).

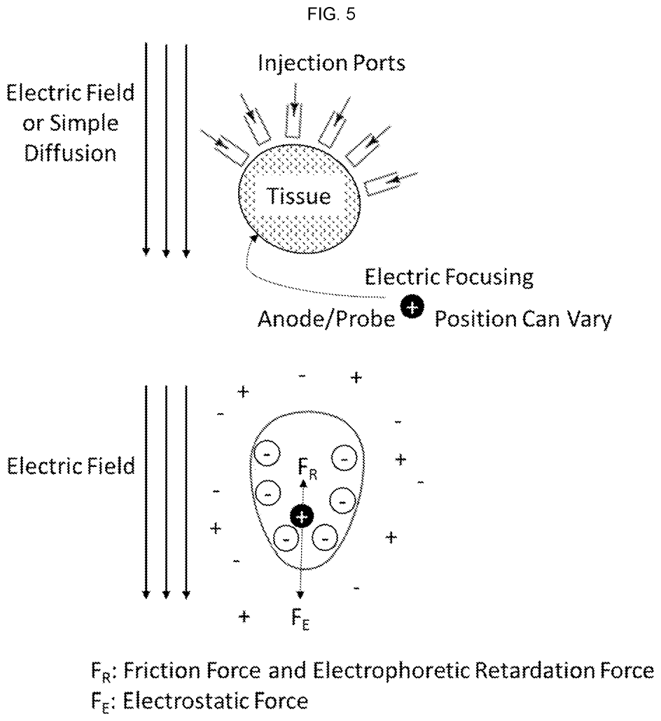

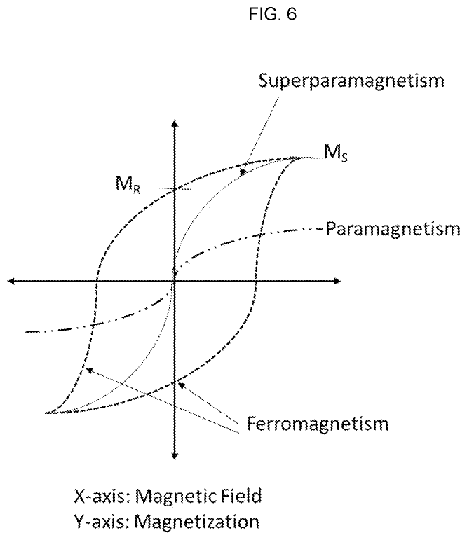

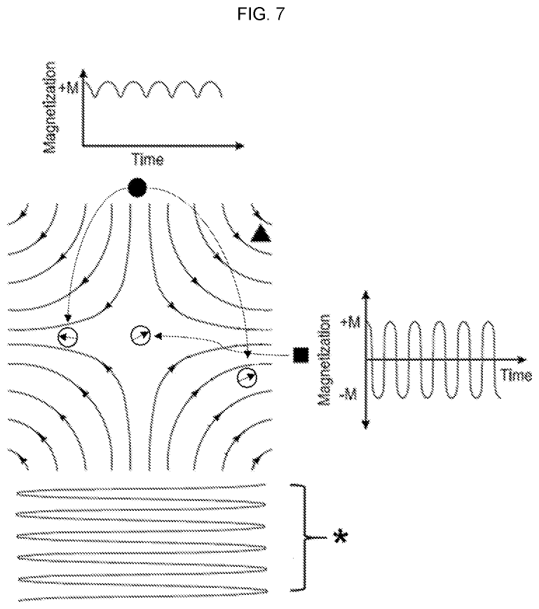

Inclusion of a ferrous metal particle as part of a spatial identifier provides additional means for adjusting the delivery direction and distance of the spatial identifiers into and within the biological sample through application of a magnetic field. The direction and strength of the magnetic field can be varied in order to provide specific delivery of the spatial identifier(s) to the desired portion of the sample, or delivery into a desired pattern within the biological sample. In addition, the type and format of any magnetic particle used as part of a spatial identifier can be selected to permit differential delivery and responsiveness to the applied magnetic field. FIGS. 5, 6, and 7 relate to the use of magnetic particles and magnetic fields in the delivery and/or positioning of spatial identifiers to and within biological samples.

In particular embodiments, spatial identifiers are delivered to a biological sample using biolistic (so called ‘gene gun’) bombardment of particles containing or coated with or otherwise associated with spatial identifiers. Methods for biolistic delivery of compounds (particularly for instance nucleic acids adsorbed on the surface of a (nano)particle such as one containing tungsten or gold or silicon) are known in the art. Biological parameters of the biological sample being analyzed (such as cell type, growth condition, and cell density) and settings on the biolistic instrumental (such as particle type and size, vacuum and pressure level, distance from the target sample) are variables that may influence the delivery of spatial identifiers using biolistic bombardment.

Biological parameters (cell type, growth condition, and cell density) and instrument settings (particle type and size, vacuum and pressure level, target distance) are variables that may impact the rate of delivery of spatial identifiers and/or their distribution through a biological sample. The art recognizes methods and systems for determining or modulating the impact (if any) of such factors on gene and other compound delivery; see, for instance, Altpeter & Sandhu (“Genetic transformation—Biolistics” in Plant Cell Culture, Wiley 2010, doi 10.1002/9780470686522.ch12); Xia et al. (“Evaluation of biolistic gene transfer methods in vivo using non-invasive bioluminescent imaging techniques” BMC Biotechnol 11:62, 2011, doi: 10.1186/1572-6750-11-62); Keshavareddy et al. (“Methods of Plant Transformation—A Review” Int J Curr Microbiol App Sci. 7(7):2656-2668, 2018), Sundowe & Reske-Kunz (“Methods in Molecular Biology—Biolistic DNA Delivery” 2013, Humana Press, Springer Science; ISBN 978-1-62703-109-7; doi 10.1007/978-1-62703-110-3); Zilony et al. (“Bombarding Cancer: Biolistic Delivery of therapeutics using Porous Si Carriers” Sci Reports 3:2499, 2013); Martin-Ortigosa & Wang (“Proteolistics: a biolistic method for intracellular delivery of proteins” Transenic Res 2014 , doi 10.1007/s11248-014-9807-y).

Also contemplated are methods and systems in which spatial identifiers are delivered to or into a biological sample using pressure shock waves or ultrasound waves. Shock wave and ultrasound applicators and methods of use thereof are known, such as those described in US20150000645 (“Shock Wave Applicator with Moveable Electrode”); US20160184526 (“Material Delivery System”); US20150174388 (“Methods and Systems for Ultrasound Assisted Delivery of a Medicant to Tissue”); Pitt et al. (“Ultrasound Drug Delivery—A General Review” Expert Opin Drug Deliv. 1(1):37-56, 2004).

It will be clear based on this description that systems and methods are envisioned in which two or more different spatial identifiers are delivered to the same biological sample. The two or more different spatial identifiers may be of the same type, may be of different types, may be delivered concurrently or sequentially, and may be delivered by the same or different methods. It is particularly contemplated that two or more different spatial identifiers are delivered into the biological sample from different application point(s) on the biological sample, such that the resultant gradient of each spatial identifier is different within the sample. Particular embodiments include more than 4 spatial identifiers, more than 8 spatial identifiers; more than 12 spatial identifiers, more than 16 spatial identifiers, or more than 20 spatial identifiers. In particular embodiments, the amount or level of a spatial identifier can include a ratio of 2 spatial identifiers, 3 spatial identifiers, 4 spatial identifiers, 5 spatial identifiers, or more. Particular embodiments utilize a time delay between delivery of different types of spatial identifiers. In particular embodiments, spatial identifiers delivered earlier in time will travel further into the biological sample than spatial identifiers delivered later in time.

Once a biological sample is labeled with one or more spatial identifiers, the sample is processed in order to enable detection and optionally quantification of label(s) and analysis that allows identification of cell type (e.g., transcriptional analysis). In general, such processing begins with disaggregation of the biological sample to produce separated cells; however, one or more additional processing steps may also be carried out, as described below.

(IV) Imaging of a Biological Sample. Particular embodiments include imaging of the biological sample before disaggregation. In particular embodiments, imaging may be carried out before or after labeling of a biological sample with spatial identifiers, or both. An image can be obtained using detection devices known in the art. Examples include microscopes configured for light, bright field, dark field, phase contrast, fluorescence, reflection, interference, or confocal imaging. A biological sample can be stained prior to imaging to provide contrast between different regions or cells. In particular embodiments, more than one stain can be used to image different aspects of the sample (e.g. different regions of a tissue, different cells, specific subcellular components or the like). In particular embodiments, a biological sample can be imaged without staining.

In particular embodiments, a fluorescence microscope (e.g. a confocal fluorescent microscope) can be used to detect a biological sample that is fluorescent, for example, by virtue of a fluorescent label. Fluorescent samples can also be imaged using a nucleic acid sequencing device having optics for fluorescent detection such as a Genome Analyzer®, MiSeq®, NextSeq® or HiSeq® platform device commercialized by Illumina, Inc. (San Diego, Calif.); or a SOLiD™ sequencing platform commercialized by Life Technologies (Carlsbad, Calif.). Other imaging optics that can be used include those that are found in the detection devices described in Bentley et al. (2008) Nature 456:53-59; WO1991/006678; WO2004/018497; WO2007/123744; U.S. Pat Nos. 7,057,026; 7,329,492; 7,211,414; 7,315,019; 7,405,281; and US20080108082.

An image of a biological sample can be obtained at a desired resolution, for example, to distinguish tissues, cells or subcellular components. Accordingly, the resolution can be sufficient to distinguish components of a biological sample that are separated by at least 0.5 μm, 1 μm, 5 pm, 10 μm, 50 μm, 100 μm, 500 μm, 1 mm or more. In particular embodiments, the resolution can be set to distinguish components of a biological sample that are separated by at least 1 mm, 500 μm, 100 μm, μm, 10 μm, 5 μm, 1 μm, 0.5 μm, or less. In particular embodiments, the distance between certain cells or cell types can be ascertained.

Methods set forth herein can include correlating location(s) in an image of a biological sample with spatial identifiers introduced to the biological sample. Accordingly, characteristics of the biological sample that are identifiable in the image can be correlated with the spatial identifiers that are found to be present in their proximity. Any of a variety of morphological characteristics can be used in such a correlation, including for example, cell shape, cell size, tissue shape, staining patterns, presence of particular proteins (e.g. as detected by immunohistochemical stains) or other characteristics that are routinely evaluated in pathology or research applications. Accordingly, in particular embodiments, the biological state of a tissue or its components as determined by visual observation can be correlated with molecular biological characteristics as determined by spatially resolved nucleic acid analysis.

A stage or support upon which a biological sample is imaged can include fiducial markers to facilitate determination of the orientation of the sample or the image thereof in relation to where the spatial identifiers are introduced onto the biological sample. Exemplary fiducials include beads (with or without fluorescent moieties or moieties such as nucleic acids to which labeled probes can be bound), fluorescent molecules attached at known or determinable features, or structures that combine morphological shapes with fluorescent moieties. Exemplary fiducials are set forth in US20020150909 and US20150125053. One or more fiducials are preferably visible while obtaining an image of a biological sample. Preferably, the stage or support includes at least 2, 3, 4, 5, 10, 25, 50, 100 or more fiducial markers. The fiducials can be provided in a pattern, for example, along an outer edge of a stage, support, or perimeter of a location where a biological sample resides. In particular embodiments, one or more fiducials are detected using the same imaging conditions used to visualize a biological sample. However, if desired, separate images can be obtained (e.g., one image of the biological sample and another image of the fiducials) and the images can be aligned to each other.

Optionally, a biological sample to be subject to a cell spatial localization method may be divided into two or more parts. Such division of the biological sample may occur before or after labeling with one or more spatial identifiers, or it may occur after one or more such spatial identifiers are applied but before at least one additional spatial identifier is applied. By way of example, division of the biological sample may include cutting the sample into two or more pieces and separating the pieces from each other, where at least one of the separated pieces is subsequently subject to additional processing (and/or labeling) steps. In some examples, the biological sample may be divided after labeling with all of the one or more spatial identifiers. When such division of the biological sample is carried out, the relative position and orientation of the various pieces to each other can be noted so that such positional information can be used in the reassembly and mapping of individual cells into their relevant spatial location upon further processing. Such relative position may be tracked using sample imaging, for instance; beneficially, such imaging may involve the use of one or more fiducial markers.

Referring to examples in which the labeled biological sample is divided into parts, it is contemplated that a biological sample will in some instances be divided into two pieces, three pieces, four pieces, five pieces, ten pieces, twenty pieces, or more; such pieces may be of equivalent size though such is not required. In some instances, the biological sample will be sliced, for instance into slices of no more than 10 cells in thickness, no more than 50 cells in thickness, or no more than 100 cells in thickness. Though not required, such sub-division of a biological sample may be useful to augment or accentuate penetration of one or more spatial identifiers into the sample.

In some examples, after the biological sample is divided into two or more parts, at least a portion of each part can be removed from that part and analyzed separately using any of the techniques described herein. For example, a portion of a face or corner of any part can be removed from that part and disagreggated. The coordinates of the portion in the biological sample can be recorded, e.g., in a machine-readable medium. Techniques described herein can then be used to determine spatial-identifier distributions of cells in the portion. The mapping from coordinates to spatial-identifier distributions can then be inverted, or used in reverse, to determine coordinates of other disaggregated cells based on their spatial-identifier distributions. For brevity, “spatial-reference data” refers to data of coordinates associated with data of spatial-identifier distributions of cells at or near those coordinates. Spatial-reference data can include data for any number of coordinates.

In some examples, including at least some examples in which the biological sample is not divided into parts, needle biopsies or other techniques described herein for removing cells from the labeled biological sample or a divided part thereof can be performed, and the cells thus removed from the labeled biological sample can be analyzed to determine their spatial-identifier distributions. Those distributions can be assembled with the coordinates from which the cells were removed to provide spatial-reference data.

In some examples, any of the techniques described above can be used with respect to a labeled phantom instead of or in addition to a labeled biological sample. Labeling, division, biopsy, or other operations on the phantom can be perform as described herein with reference to biological samples. In some examples, the phantom has substantially the same size, shape, or density as the biological sample. Spatial-reference data can then be determined as noted above using the data from the phantom.

In particular embodiments, the dividing or biopsy of a biological sample or labeled phantom can ultimately provide spatial-reference data that can serve as calibrated reference points for locations of other cells within the biological sample due to knowledge of location of the cells in proximity to or at a cut or biopsy.

In some examples, mathematical models of the transport of the spatial identifiers through the biological sample can be used instead of or in addition to measurements of the sample or of a phantom to provide spatial-reference data. For example, models of biological diffusion, fluidic, or transport processes can be used to estimate the spatial-identifier distributions at various points in a simulation of the biological sample. The estimated spatial-identifier distributions and the points at which they are estimated can be assembled into spatial-reference data.

In any of the examples above of determining spatial-reference data, the spatial-reference data can be determined, e.g., at random (or pseudo-random) coordinates; on a grid having particular grid spacings in one or more dimensions; at substantially uniformly-spread coordinates determined in other ways, e.g., using a diffusion pattern such as blue-noise diffusion; at substantially non-uniformly-spread coordinates, e.g., more closely spaced in areas where higher spatial resolution is required; or any combination of any of those. In some examples, using a finer grid (smaller grid spacing(s)) can provide more accurate spatial maps than using a coarser grid (larger grid spacing(s)).

In particular embodiments, spatial-reference data is used in determining the 3D spatial model of position. In an illustrative example, the spatial-reference data can include a first coordinate, an associated first spatial-identifier distribution, a second coordinate, and an associated second spatial-identifier distribution. The first and second coordinates can be different. Each spatial-identifier distribution can include a respective concentration of a particular spatial identifier. Linear regression can be used based on the spatial-reference data [(concentration 1, coordinates 1), (concentration 2, coordinates 2)] to determine a linear function from concentration of the spatial identifier to position. Then, for any disaggregated cell, the concentration of the particular spatial identifier can be mapped through the linear function to determine the estimated coordinates of that cell's location in the labeled biological sample.

In particular embodiments, regression or other fitting techniques can be used to fit functions from any number of independent variables (e.g., one per spatial identifier) to any number of dependent variables (e.g., 1-3 dependent variables, representing respective coordinates in a 3D space). Regression or fitting models that can be used can include linear, nonlinear, logistic, polynomial, exponential, or other models.

In particular embodiments, multiple regression or fitting models can be determined. For example, three spatial identifiers can be used, each injected or otherwise added to the biological sample along one of three orthogonal axes in a coordinate system having an origin within the biological sample. Three fits can be performed: one to map concentration of the first identifier to the X coordinate, one to map concentration of the second identifier to the Y coordinate, and one to map concentration of the third identifier to the Z coordinate.

In particular embodiments, multiple spatial identifiers can be used on each axis. For example, two different spatial identifiers can be added to the biological sample on the +X and −X axes, respectively. A fit can then be determined from the concentrations of those two values to the X coordinate. This can improve coordinate accuracy in the case of nonuniform diffusion of a spatial identifier along the ±X axis.

In particular embodiments, models mapping spatial-identifier distributions to coordinates, in any of the combinations of identifiers and coordinates above or in other combinations, can be determined using computational models such as neural networks, decision trees, or other machine-learning models. The spatial-reference data can be used as training data, or can be divided into training, test, or validation datasets. The training data can be used to train a computational model (CM) to take spatial-identifier distributions as inputs and produce coordinates as outputs.

In particular embodiments, the CMs may include one or more regression models, e.g., polynomial and/or logistic regression models; classifiers such as binary classifiers; decision trees, e.g., boosted decision trees, configured for, e.g., classification or regression; and/or artificial neurons, e.g., interconnected to form a multilayer perceptron or other neural network. A decision tree can include, e.g., parameters defining hierarchical splits of a feature space into a plurality of regions. A decision tree can further include associated classes, values, or regression parameters associated with the regions. A neural network (NN) can have none, at least one, or at least two hidden layers. NNs having multiple hidden layers are referred to as deep neural networks (DNNs).

In particular embodiments, CMs can include one or more recurrent computational models (RCMs). An RCM can include artificial neurons interconnected so that the output of a first unit can serve as a later input to the first unit and/or to another unit not in the layer immediately following the layer containing the first unit.

At least one of the computational model(s) can include, e.g., activation weights, functions, and/or thresholds for artificial neurons and/or other computational units (e.g., long short-term memory units) of one or more neural networks; coefficients of learned ranking functions, e.g., polynomial functions; and/or parameters of decision trees and/or other classifiers, in some nonlimiting examples. These are referred to individually or collectively as “parameters” herein.

A modeling engine can be configured to determine CMs, e.g., to apply NN-training techniques to determine neuron parameters of artificial neurons in the CMs. For example, modeling engines can determine CMs using a reinforcement-learning update rule. The modeling engine can parallelize the training of the NNs and/or other determination algorithms for CMs across multiple processing units, e.g., cores of a multi-core processor and/or multiple general-purpose graphics processing units (GPGPUs). For example, multiple layers of DNNs may be processed in parallel on the multiple processing units. The modeling engine can train neural networks such as DNNs minibatch-based stochastic gradient descent (SGD). SGD can be parallelized along, e.g., model parameters, layers, and data (and combinations thereof). Other frameworks besides SGD can be used, e.g., minibatch non-stochastic gradient descent and/or other mathematical-optimization techniques. The modeling engine can determine CMs at least in part using an experience replay or “bag-of-transitions” reinforcement-learning update rule.

In particular embodiments, CMs can be determined and/or adjusted using the Theano and/or scikit-learn packages for PYTHON, and/or another symbolic/numerical equation solver, e.g., implemented in C++, C#, MATLAB, Octave, and/or MATHEMATICA.

In particular embodiments, a learning-step function can be given as input a randomly-selected minibatch of the training data at each call in order to determine and/or adjust the model according to stochastic gradient descent (SGD) techniques. In some examples, computational models can be determined or adjusted using SGD with momentum. Grid search can be used to select the learning rate for, e.g., NN training and/or other CM determination and/or adjustment. Alternatively, several candidate policies can be determined using various learning rates and the candidate policy best satisfying acceptance criteria, e.g., of accuracy and/or precision, can be selected.

In particular embodiments, a CM can be determined using one or more minibatches of the training data, e.g., a plurality of minibatches. A CM can be determined in a stochastic manner, e.g., by selecting minibatches at random from logging data. In some examples, using stochastic techniques can provide improved speed of convergence and/or improved numerical stability of the CM-determination process. In some examples, minibatches can be processed in parallel to reduce training time.

(V) Disaggregation of a Labeled Biological Sample. In order to permit cell-by-cell identification and analysis of the spatial identifiers, cells of a biological sample (or sub-samples) that have been labeled are disaggregated or dissociated from each other using any one of myriad well known and recognized techniques. The methods used for disaggregation (or dissociation) may be influenced by a number of factors, including the type of cells under analysis (animal, plant, fungal, etc.), the type of spatial identifier(s) used (fluorescent, targeted, radioactive, etc.), the type of analysis intended to be performed after disaggregation (flow cytometry, sequencing, electromagnetic imagery, etc.), and so forth. In general, any disaggregation method could be used so long as it produces a majority of separated cells that are intact as long as needed and/or maintain surface labeling (if a surface-targeted spatial identifier was employed), and which preserves the molecule(s) used as spatial identifiers in a manner sufficient for further analysis of those molecules as they are associated with the disaggregated cells. In particular embodiments, disaggregation or dissociation of a biological sample yields a suspension of cells. In particular embodiments, the cells from the suspension can be partitioned into individual compartments, each compartment containing a single cell, as described herein.

By way of example, mechanical disaggregation or enzymatic disaggregation can be used to process biological samples after spatial identifier labeling. Generally, enzymatic disaggregation (which relies on chemical degradation of one or more of the components surrounding cells or holding cells together) is more reliable and will provide a higher yield of disaggregated cells. For enzymatic disaggregation of cells from an animal tissue, trypsin, collagenase, pronase, dispase, hyaluronidase, thermolysin, neuraminidase, or a mixture of two or more thereof can be used. Commercial preparations of enzymes useful in animal cell disaggregation are available, including for instance: Dispase II, powder (ThermoFisher Catalog #17105041); Collagenase, Type I, powder (ThermoFisher Catalog #s 17100017 and 17018029); Collagenase, Type II, powder (ThermoFisher Catalog # 17101015); Collagenase, Type IV, powder (ThermoFisher Catalog # 17104019); Trypsin (ThermoFisher Catalog #s15050057, 15050065, and 15090046); Iiberase™ TL (low Thermolysin concentration, Sigma Millipore, Burlington, Mass.); and TrypLE™ Express Enzyme (a substitute for trypsin; cleaves peptide bonds on the C-terminal side of lysine and arginine (ThermoFisher Catalog #s 12604013, 12604021, 12605010, and 12605028).

For enzymatic disaggregation of cells from plants, different enzymes are applicable; these include pectinase, cellulase, hemicellulase, and lignase or mixtures of two or more thereof. For enzymatic disaggregation of fungal cells, chitinase, glucanases, and proteinases are useful.

Art-known techniques for tissue disaggregation or dissociation can be found, for instance, in the following: Cunningham, Methods Mol Biol 588:327-330, 2010.

In particular embodiments, one or more spatial identifiers associated with a given cell from a dissociated biological sample can be subjected to sequencing as described herein to yield sequencing reads that can identify the location of the cell within the biological sample.

(VI) Sequestration of Cells from a Labeled Biological Sample. In various embodiments, it can be useful to sequester disaggregated cells from each other. This is particularly useful to ensure that subsequent analysis steps regarding cell type identity provide results linked to individual cells. Cells can be sequestered into individual containers, such as the wells of microplates; into isolation droplets of various compositions (including gel or oil droplets); and so forth. In particular embodiments, sequestration of cells includes compartmentalizing or partitioning each cell into its own separate container (e.g., microplate well, droplet). In particular embodiments, compartmentalization or partitioning of single cells allows association (linkage) of a single barcoded oligonucleotide with genomic and/or transcriptomic sequences of a single cell.

Methods for delivering individual cells into microplate wells or other containers are known in the art. These include, for instance, flow cytometry (see, for instance, Ibrahim & van den Engh, Adv Biochem Eng Biotechnol. 106:19-39, 2007). In those embodiments where one or more of the spatial identifiers include a fluorescent moiety, labeled disaggregated cells can also be segregated using fluorescence activated cell sorting (FACS) or similar techniques, which use flow cytometry coupled with sensing of the fluorescent labeling of cells to disperse cells into different locations.

Also contemplated are methods of sequestering individual cells into droplets or microdroplets, for instance as described in Zhang et al. (Scientific Reports 7: 41192, 2017), Terekhov et al. (PNAS USA, 114(10):2550-2555, 2017); Brouzes (Methods Mol Biol. 853:105-139, 2012); US20170260584. Specifically, the gel microbead sequestration techniques used with single cell RNA-Seq are contemplated for use with methods provided herein.

In certain cases, microfluidic channel networks are particularly suited for generating partitions as described herein. Alternative mechanisms may also be employed in the partitioning of individual cells, including porous membranes through which aqueous mixtures of cells are extruded into non-aqueous fluids. Such systems are generally available from, e.g., Nanomi, Inc.

In various embodiments, compartments include droplets of aqueous fluid within a non-aqueous continuous phase, e.g., an oil phase. In alternative embodiments, compartments can refer to containers or vessels (such as wells, microwells, tubes, through ports in nanoarray substrates, or other containers). These compartments may include, e.g., microcapsules or micro-vesicles that have an outer barrier surrounding an inner fluid center or core, or they may be a porous matrix that is capable of entraining and/or retaining materials within its matrix. A variety of different vessels are described in, for example, US20140155295. Likewise, emulsion systems for creating stable droplets in non-aqueous or oil continuous phases are described in detail in, e.g., US20100105112.

In the case of droplets in an emulsion, allocating individual cells to discrete compartments may generally be accomplished by introducing a flowing stream of cells in an aqueous fluid into a flowing stream of a non-aqueous fluid, such that droplets are generated at the junction of the two streams. By providing the aqueous cell-containing stream at a certain concentration level of cells, the level of occupancy of the resulting partitions in terms of numbers of cells can be controlled. In some cases, where single cell partitions are desired, it may be desirable to control the relative flow rates of the fluids such that, on average, the partitions contain less than one cell per partition, in order to ensure that those partitions which are occupied, are primarily singly occupied.

Although described in terms of providing substantially singly occupied partitions, above, in certain cases, it is desirable to provide multiply occupied partitions, e.g., containing two, three, four or more cells within a single partition. Accordingly, as noted above, the flow characteristics of the cell and/or bead containing fluids and partitioning fluids may be controlled to provide for such multiply occupied partitions.

The partitions described herein can be characterized by having extremely small volumes, e.g., less than 10 microliters (μL), 5 μL, 1 μL, 900 nanoliters (nL), 500 nL, 100 nL, 50 nL, 1 nL, 900 picoliters (pL), 800 pL, 700 pL, 600 pL, 500 pL, 400 pL, 300 pL, 200 pL, 100 pL, 50 pL, 20 pL, 10 pL, or 1 pL. For example, in the case of droplet based partitions, the droplets may have overall volumes that are less than 1000 pL, 900 pL, 800 pL, 700 pL, 600 pL, 500 pL, 400 pL, 300 pL, 200 pL, 100 pL, 50 pL, 20 pL, 10 pL, or even less than 1 pL.

Multiple samples can be processed in parallel using droplet based systems disclosed herein. In some embodiments, at least 2, 3, 4, 5, 6, 7, 8, 9, or 10 samples are processed in parallel. The multiple samples processed in parallel may include similar numbers of cells. In some cases, the multiple samples processed in parallel do not include similar numbers of cells.

A cell population for analysis can include any number of cells. In some embodiments, a cell sample loaded on a droplet based system includes at least 100, 1,000, 10,000, 20,000, 30,000, 40,000, 50,000, 60,000, 70,000, 80,000, 90,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, or at least 1,000,000 cells.

(VII) Cellular Labels. Once separated using any appropriate cell sorting technique, or during the separating process, a cell can be associated with a cellular label that identifies its individual partition following biological sample disaggregation. Similar to spatial identifiers, a cellular label can be any unique identifier (e.g., barcode), that is previously, subsequently or concurrently delivered to a partition that holds a compartmentalized or partitioned cell. Cellular labels that include a barcode sequence, may be delivered, in some embodiments, on an oligonucleotide (referred to interchangeably as a “barcoded oligonucleotide” or “oligonucleotide barcode”), to a partition via any suitable mechanism. In some cases, it may be desirable to incorporate multiple different cellular labels within a given partition, either attached to a single bead or to multiple beads within the partition. For example, in some cases, a mixed, but known barcode sequence set may provide greater assurance of identification in the subsequent processing, e.g., by providing a stronger address or attribution of the barcodes to a given partition, as a duplicate or independent confirmation of the output from a given partition.

In particular embodiments, barcoded oligonucleotides are delivered to a partition via a microcapsule. In some cases, barcoded oligonucleotides are initially associated with the microcapsule and then released from the microcapsule upon application of a stimulus which allows the oligonucleotides to dissociate or to be released from the microcapsule.

A microcapsule, in some embodiments, includes a bead. In some embodiments, a bead may be porous, non-porous, solid, semi-solid, semi-fluidic, or fluidic. In some embodiments, a bead may be dissolvable, disruptable, or degradable. In some cases, a bead may not be degradable. In some embodiments, the bead may be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the beads are silica beads. In some cases, the beads are rigid. In some cases, the beads are flexible and/or compressible.

The beads may contain molecular precursors (e.g., monomers or polymers), which may form a polymer network via polymerization of the precursors. In some cases, a precursor may be an already polymerized species capable of undergoing further polymerization via, for example, a chemical cross-linkage. In some cases, a precursor includes one or more of an acrylamide or a methacrylamide monomer, oligomer, or polymer. In some cases, the bead may include prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they include mixed polymers, co-polymers, and/or block co-polymers. For additional options, see US20170260584.

in particular embodiments, beads are provided that each include large numbers of the above described oligonucleotides releasably attached to the beads, where all of the oligonucleotides attached to a particular bead will include the same nucleic acid barcode sequence, but where a large number of diverse barcode sequences are represented across the population of beads used. In particularly useful examples, gel beads are used as a solid support and delivery vehicle for the oligonucleotides into the partitions, as they are capable of carrying large numbers of oligonucleotide molecules and may be configured to release those oligonucleotides upon exposure to a particular stimulus, as described below. In some cases, the population of beads will provide a diverse barcode sequence library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences. Additionally, each bead can be provided with large numbers of oligonucleotide molecules attached. In particular, the number of molecules of oligonucleotides including the barcode sequence on an individual bead can be at least 1,000 oligonucleotide molecules, at least 5,000 oligonucleotide molecules, at least 10,000 oligonucleotide molecules, at least 50,000 oligonucleotide molecules, at least 100,000 oligonucleotide molecules, at least 500,000 oligonucleotides, at least 1,000,000 oligonucleotide molecules, at least 5,000,000 oligonucleotide molecules, at least 10,000,000 oligonucleotide molecules, at least 50,000,000 oligonucleotide molecules, at least 100,000,000 oligonucleotide molecules, and in some cases at least 1 billion oligonucleotide molecules.

The oligonucleotides can be releasable from the beads upon the application of a particular stimulus to the beads. In some cases, the stimulus may be a photo-stimulus, e.g., through cleavage of a photo-labile linkage that releases the oligonucleotides. In some cases, a thermal stimulus may be used, where elevation of the temperature of the beads environment will result in cleavage of a linkage or other release of the oligonucleotides form the beads. In some cases, a chemical stimulus is used that cleaves a linkage of the oligonucleotides to the beads, or otherwise results in release of the oligonucleotides from the beads. Examples of this type of system are described in US20140155295 and US20140378345. In particular embodiments, such compositions may be degraded for release of the attached oligonucleotides through exposure to a reducing agent, such as DTT. Various combinations of the stimuli may also be used to trigger cleavage of different oligos at different times within a process.

In accordance with certain aspects, the cells may be partitioned along with additional reagents, such as lysis reagents in order to release the contents of the cells within the partition. In such cases, the lysis agents can be contacted with the cell suspension concurrently with, or immediately prior to the introduction of the cells into the partitioning junction/droplet generation zone, e.g., through an additional channel or channels upstream of the channel junction. Examples of lysis agents include bioactive reagents, such as lysis enzymes that are used for lysis of different cell types, e.g., gram positive or negative bacterial cells, plant cells, yeast cells, mammalian cells, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the cells to cause the release of the cell's contents into the partitions. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Similarly, lysis methods that employ other methods may be used, such as electroporation, thermal, acoustic or mechanical cellular disruption may also be used in certain cases, e.g., non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition. In some cases, this stimulus may be the same as the stimulus described elsewhere herein for release of oligonucleotides from their respective bead or partition. In alternative aspects, this may be a different and non-overlapping stimulus, in order to allow an encapsulated cell to be released into a partition at a different time from the release of oligonucleotides into the same partition.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides (also referred to herein as “switch oligos”) which can be used for template switching. In some cases, template switching can be used to increase the length of a cDNA. In one example of template switching, cDNA can be generated from reverse transcription of a template, e.g., cellular mRNA, where a reverse transcriptase with terminal transferase activity can add additional nucleotides, e.g., polyC, to the cDNA that are not encoded by the template, such, as at an end of the cDNA. Switch oligos can include sequences complementary to the additional nucleotides, e.g. polyG. The additional nucleotides (e.g., polyC) on the cDNA can hybridize to the sequences complementary to the additional nucleotides (e.g., polyG) on the switch oligo, whereby the switch oligo can be used by the reverse transcriptase as a primer to generate a polynucleotide sequence complementary to the cDNA. Switch oligos may include deoxyribonucleic acids, ribonucleic acids, modified nucleic acids including locked nucleic acids (LNA), or any combination.

In some cases, the length of a switch oligo may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 nucleotides or longer.

Once the contents of the cells are released into their respective partitions, the nucleic acids contained therein may be further processed within the partitions including, e.g., fragmentation, amplification and barcoding, as well as attachment of other functional sequences. As noted above, fragmentation may be accomplished through the co-partitioning of shearing enzymes, such as endonucleases, in order to fragment the nucleic acids into smaller fragments. These endonucleases may include restriction endonucleases, including type II and type Ils restriction endonucleases as well as other nucleic acid cleaving enzymes, such as nicking endonucleases, and the like. In some cases, fragmentation may not be desired, and full-length nucleic acids may be retained within the partitions, or in the case of encapsulated cells or cell contents, fragmentation may be carried out prior to partitioning, e.g., through enzymatic methods, e.g., those described herein, or through mechanical methods, e.g., mechanical, acoustic or other shearing.

As indicated, once co-partitioned, and the cells are lysed to release their nucleic acids, the oligonucleotides disposed upon the bead may be used to barcode and amplify fragments of those nucleic acids. A particularly elegant process for use of these barcode oligonucleotides in amplifying and barcoding fragments of sample nucleic acids is described in detail in US20140378345. Briefly, in one aspect, the oligonucleotides present on the beads that are co-partitioned with the cells, are released from their beads into the partition with the cell's nucleic acids. The oligonucleotides can include, along with the barcode sequence, a primer sequence at its 5′ end. This primer sequence may be a random oligonucleotide sequence intended to randomly prime numerous different regions on the cell's nucleic acids, or it may be a specific primer sequence targeted to prime upstream of a specific targeted region of the cell's genome.

Once released, the primer portion of the oligonucleotide can anneal to a complementary region of the cell's nucleic acid. Extension reaction reagents, e.g., DNA polymerase, nucleoside triphosphates, co-factors (e.g., Mg2+ or Mn2+), that are also co-partitioned with the cells and beads, then extend the primer sequence using the cell's nucleic acid as a template, to produce a complementary fragment to the strand of the cell's nucleic acid to which the primer annealed, which complementary fragment includes the oligonucleotide and its associated barcode sequence. Annealing and extension of multiple primers to different portions of the cell's nucleic acids will result in a large pool of overlapping complementary fragments of the nucleic acid, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow formation of a hairpin structure or partial hairpin structure, the reduces the ability of the molecule to be the basis for producing further iterative copies.

In accordance with the methods and systems described herein, the nucleic acid contents of individual cells are generally provided with unique identifiers such that, upon characterization of those nucleic acids they may be attributed as having been derived from the same cell or cells. The ability to attribute characteristics to individual cells or groups of cells is provided by the assignment of unique identifiers specifically to an individual cell or groups of cells, which is another advantageous aspect of the methods and systems described herein. In particular, unique identifiers, e.g., in the form of nucleic acid barcodes are assigned or associated with individual cells or populations of cells, in order to tag or label the cell's components (and as a result, its characteristics) with the unique identifiers. These unique identifiers are then used to attribute the cell's components and characteristics to an individual cell or group of cells. In some aspects this is carried out by co-partitioning the individual cells or groups of cells with the unique identifiers. In some aspects the unique identifiers are provided in the form of oligonucleotides that include nucleic acid barcode sequences that may be attached to or otherwise associated with the nucleic acid contents of individual cells, or to other components of the cells, and particularly to fragments of those nucleic acids. The oligonucleotides are partitioned such that as between oligonucleotides in a given partition, the nucleic acid barcode sequences contained therein are the same, but as between different partitions, the oligonucleotides can, and do have differing barcode sequences, or at least represent a large number of different barcode sequences across all of the partitions in a given analysis. In some aspects only one nucleic acid barcode sequence can be associated with a given partition, although in some cases, two or more different barcode sequences may be present. In particular embodiments, nucleic acid barcodes can be distinct from UMIs. In particular embodiments, each nucleic acid barcode can be used to identify the cell type of a single cell in a biological sample, whereas a UMI can be used to quantify cDNA originating from different mRNA molecules of a given partitioned cell.

In particular embodiments, a single cell can be partitioned with a cellular label including a barcoded oligonucleotide. The partitioned cell includes a first set of polynucleotides that can be subjected to nucleic acid amplification using extension reaction reagents described above to generate a second set of polynucleotides. In particular embodiments, the second set of polynucleotides includes: (i) a segment having a sequence of a polynucleotide of the first set of polynucleotides, and (ii) a segment having a sequence of a cellular label including a nucleic acid barcode. A library of second sets of polynucleotides can be generated from a plurality of partitions, each partition including a single cell of a biological sample and a distinct cellular label including a barcoded oligonucleotide. The library can then be subjected to sequencing as described herein to yield sequencing reads that can identify each cell within a biological sample as having a particular cell type according to the cellular label associated with each cell.

As with nucleic acid barcode sequences used as spatial identifiers, the nucleic acid barcode sequences used as cellular labels and/or unique molecular identifiers can be any of a variety of lengths. Longer sequences can generally accommodate a larger number and variety of barcodes. All barcoded nucleic acids in a plurality can have the same length barcode (albeit with different sequences), but it is also possible to use different length barcodes in different nucleic acids. A barcode sequence can be at 4, 6, 7, 8, 9, 10, 11, 12, 13, 14, 15, 16, 17, 18, 19, 20 nucleotides or longer. In particular embodiments, a barcode sequence may have a length in range of from 6 to 250 nucleotides, from 6 to 130 nucleotides, from 6 to 30 nucleotides, from 8 to 120 nucleotides or from 8 to 100 nucleotides. These nucleotides may be completely contiguous, i.e., in a single stretch of adjacent nucleotides, or they may be separated into two or more separate subsequences that are separated by 1 or more nucleotides. Barcode sequences are described in, for example: U.S. Pat. No. 5,635,400; Brenner et al., Proc. Natl. Acad. Sci., 97:1665-1670, 2000; Shoemaker et al., Nature Genetics 14: 450-456, 1996; EP0799897; U.S. Pat. No. 5,981,179; US20140342921; and U.S. Pat. No. 8,460,865.

The single cell analysis methods described herein, including the partition of cells, cellular labeling, amplification of nucleic acids within the cells, and sequencing of amplified nucleic acids can be used to generate a genetic profile for a given cell. In particular embodiments, a genetic profile can include a genomic profile that includes sequence information of a portion or all of the genes, coding regions, and/or noncoding regions in the genome of a given cell. In particular embodiments, the amplification of the cell's nucleic acids is carried out until the barcoded overlapping fragments within the partition constitute at least 1× coverage of the particular portion or all of the cell's genome, at least 2×, at least 3×, at least 4×, at least 5×, at least 10×, at least 20×, at least 40× or more coverage of the genome or its relevant portion of interest. Once the barcoded fragments are produced, they may be directly sequenced on an appropriate sequencing system, e.g., an Illumina Hiseq®, Miseq® or X10 system, or they may be subjected to additional processing, such as further amplification, attachment of other functional sequences, e.g., second sequencing primers, for reverse reads, sample index sequences, and the like.

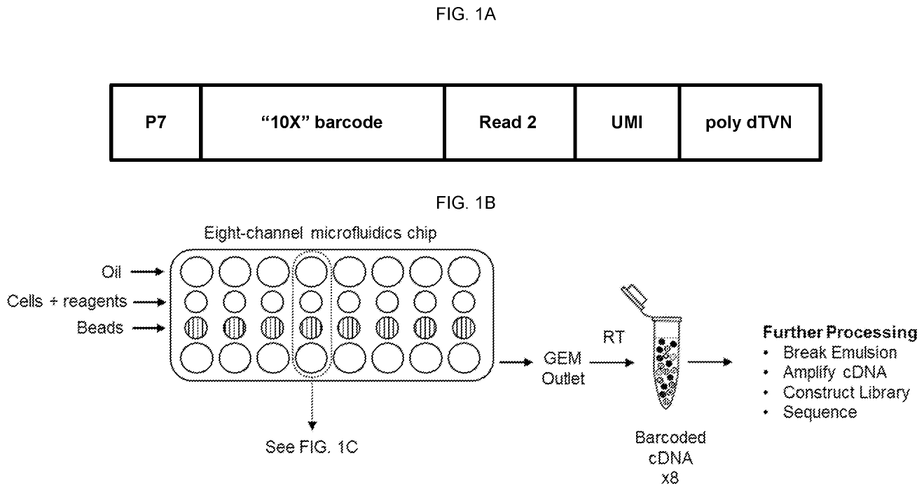

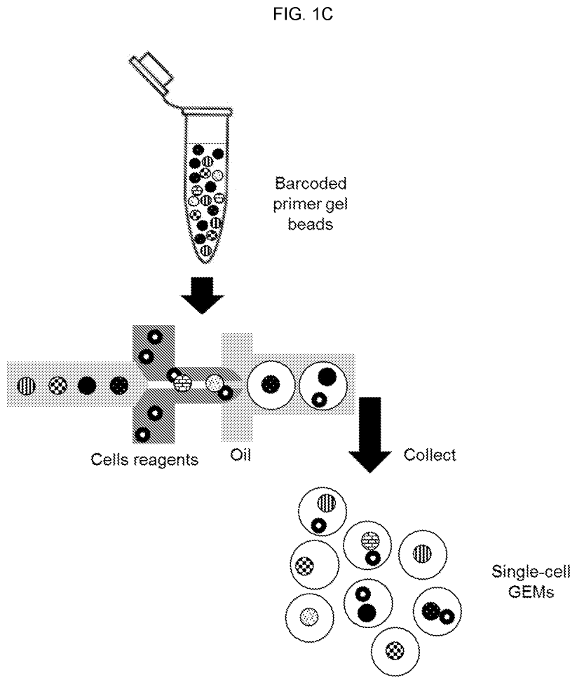

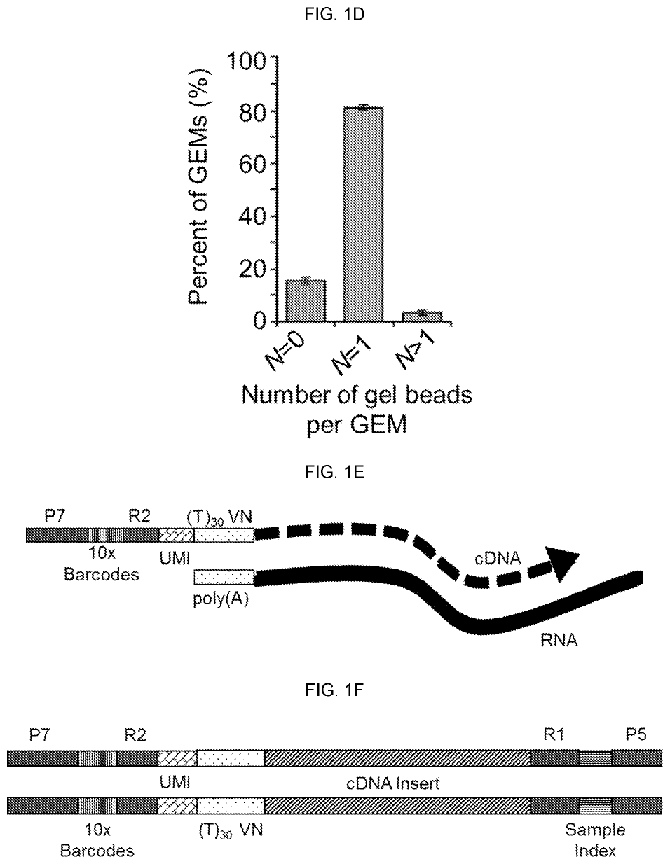

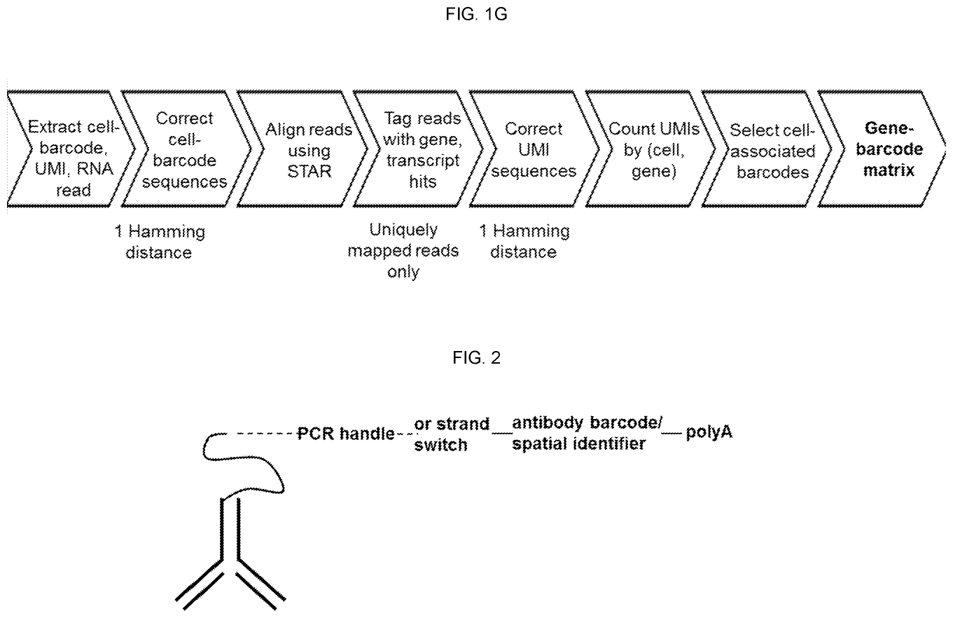

The single cell analysis methods described herein may also be useful in the analysis of the transcriptome of a cell, which includes the set of all RNA molecules in one cell. In particular embodiments, the transcriptome can refer to all RNAs in a cell. In particular embodiments, the transcriptome can refer to all mRNA in a cell. In particular embodiments, a genetic profile can include a transcriptomic profile that includes gene expression for all or a portion of the genes in the genome of a given cell. In an example method of whole transcriptome analysis using the single cell analysis methods described herein, an individual cell is co-partitioned along with a bead bearing a barcoded oligonucleotide that includes a poly-T sequence, and other reagents such as reverse transcriptase, polymerase, a reducing agent, a switch oligo, and dNTPs into a partition (e.g., droplet in an emulsion). In an operation of this method, the cell is lysed while the barcoded oligonucleotides are released from the bead (e.g., via the action of the reducing agent) and the poly-T sequence hybridizes to the poly-A tail of cellular mRNA. In a reverse transcription reaction using the mRNA as template, cDNA transcripts of cellular mRNA can be produced (see, e.g., FIG. 1E). The RNA can then be degraded with an RNase. Next, the poly-T segment is extended in a reverse transcription reaction using the mRNA as a template to produce a first strand cDNA complementary to the mRNA and also includes the barcoded oligonucleotide. Terminal transferase activity of the reverse transcriptase can add additional bases to the first strand cDNA (e.g., polyC). The switch oligo may then hybridize with the additional bases added to the first strand cDNA and facilitate template switching from the mRNA to the first strand cDNA. A sequence complementary to the first strand cDNA, the second strand cDNA can then be generated via extension of the switch oligo using the first strand cDNA as a template. Within any given partition, all of the cDNA of the individual mRNA molecules can include a common barcode sequence segment. This common barcode segment serves as a transcriptional label for a cell (“transcriptional label nucleic acid barcode”). However, by including a unique random N-mer sequence (UMI), cDNA made from different mRNA molecules within a given partition will vary at this unique sequence. This provides a quantitation feature that can be identifiable even following any subsequent amplification of the contents of a given partition, e.g., the number of unique segments associated with a common barcode can be indicative of the quantity of mRNA originating from a single partition, and thus, a single cell. Following second strand cDNA synthesis, the cDNA can be amplified with primers to yield a product that includes a plurality or totality of cDNA molecules in a given cell flanked by sequences including a barcode that identifies a cell, sequences to prime sequencing, a sample index sequence, and/or a UMI (see, e.g., FIG. 1F). The functional sequences of a barcoded oligonucleotide may include: a sequencer specific flow cell attachment sequence, e.g., a P5 or P7 sequence for Illumina sequencing systems; a sequencing primer binding site, e.g., for a R1 or R2 primer for Illumina sequencing systems; a UMI; a sample index, e.g., an i7 sample index sequence for Illumina sequencing systems. In particular embodiments where a partition is a droplet in an emulsion, the emulsion can be broken and the contents of the droplet pooled in order to complete amplification of cDNA. In particular embodiments, cDNA amplification may be completed in the partition.

Functional sequences may be selected to be compatible with a variety of different sequencing systems, e.g., 454 Sequencing, Ion Torrent Proton or PGM, Illumina X10, etc., and the requirements thereof. Particular embodiments perform transcriptional analysis as described in US20170260584 to allow identification of particular cell types.

Additional sequencing techniques may also be utilized so long as they allow a cell to retain its linked relationship with at least its spatial identifier and cellular (e.g., genomic and/or transcriptional) label. For example, the sequencing-by-synthesis (SBS) technique is a particularly useful method for determining barcode sequences. SBS can be carried out as follows: To initiate a first SBS cycle, one or more labeled nucleotides, DNA polymerase, SBS primers etc., can be contacted with a nucleic acid to be sequenced. A labeled nucleotide incorporated during SBS primer extension can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is encountered to remove the moiety. Thus, for embodiments that use reversible termination, a deblocking reagent can be delivered during sequencing (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be adapted for use with systems and methods of the present disclosure are described, for example, in Bentley et al., Nature 456:53-59, 2008; WO1991/006678; WO2004/018497; WO2007/123744; U.S. Pat. Nos. 7,057,026; 7,329,492; 7,211,414; 7,315,019; 7,405,281; and US20080108082.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi et al., Analytical Biochemistry 242(1): 84-89, 1996; Ronaghi, Genome Res. 11 (1), 3-11, 2001; Ronaghi et al., Science 281(5375): 363, 1998; U.S. Pat. Nos. 6,210,891; 6,258,568; and 6,274,320). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be used for application of pyrosequencing to systems and methods of the present disclosure are described, for example, in WO2012/058096; US20050191698; U.S. Pat. Nos. 7,595,883; and 7,244,559.

Sequencing-by-ligation reactions are also useful including, for example, those described in Shendure et al., Science 309:1728-1732, 2005; U.S. Pat. Nos. 5,599,675; and 5,750,341.

Some sequencing embodiments can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al., Science 299: 682-686, 2003; Lundquist et al., Opt. Lett. 33: 1026-1028, 2008; and Korlach et al., Proc. Natl. Acad. Sci. USA 105: 1176-1181, 2008.

Some sequencing embodiments include detection of a proton released upon incorporation of a nucleotide into an extension product. For example, sequencing based on detection of released protons can use an electrical detector and associated techniques that are commercially available from Ion Torrent (Guilford, Conn.; a Life Technologies and Thermo Fisher subsidiary) or sequencing methods and systems described for instance in US20090026082; US20090127589; US20100137143; and US20100282617.

Once a biological sample has been labeled with one or more spatial identifiers, labeled cells within the biological sample are disaggregated such that individual cells can be separately analyzed. The presence and/or level of individual label(s) in a plurality of cells can be determined by sequencing, and characteristics of the presence and/or level of labels in cells can be used to determine the spatial location of individual cells or groups of cells in the original biological sample. In particular embodiments, the presence, amount, or type of spatial identifier nucleic acid barcode sequences allow assignment of a spatial location of the partitioned cell before biological sample disaggregation. In particular embodiments, cellular label nucleic acid barcode sequences of the plurality of cellular label nucleic acid barcode sequences associate sequencing reads with individual partitioned cells. In particular embodiments, cellular label nucleic acid barcode sequences allow identification of a cell type for a given partitioned cell. In particular embodiments, the cellular label nucleic acid barcodes are transcriptional label nucleic acid barcodes. In particular embodiments, processing sequencing data associated with spatial identifier nucleic acid barcode sequences and/or cellular label nucleic acid barcode sequences enable the clustering of cells having identical characteristics of a spatial identifier nucleic acid barcode (e.g., presence, amount, particular sequence) and/or identical characteristics of a cellular label nucleic acid barcode (e.g., presence, amount, particular sequence).

In particular embodiments, the disclosure provides determining a percentage of the heterogeneous cell sample represented by the minor cell population and/or the major cell population. The percentage of the heterogeneous cell sample represented by the minor cell population can be determined at a sensitivity of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The percentage of the heterogeneous cell sample represented by the major cell population can be determined at a sensitivity of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Returning to the library of second sets of polynucleotides described above that can be generated from amplification of nucleic acids from a plurality of partitions, sequencing of cellular labels (genomic and/or transcriptional labels) associated with individual partitioned cells can generate (i) a first set of genetic profiles corresponding to a first cell type, and (ii) a second set of genetic profiles corresponding to a second cell type, which first and second set of genetic profiles differentiate a cell of the first cell type from a cell of the second cell type. As mentioned above, a set of genetic profiles can include genomic and/or transcriptomic profiles. Single nucleotide variants (SNVs) can be used to distinguish a first set of genetic profiles for a given cell from a second set of genetic profiles for a given different cell. In particular embodiments, SNVs can include a substitution of a single nucleotide of a given sequence with another nucleotide relative to a reference set of sequences. In particular embodiments, a set of genetic profiles can include 1 SNV, 2 SNVs, 3 SNVs, 4 SNVs, 5 SNVs, 6 SNVs, 7 SNVs, 8 SNVs, 9 SNVs, 10 SNVs, 15 SNVs, 20 SNVs, 25 SNVs, 30 SNVs, 35 SNVs, 40 SNVs, 45 SNVs, 50 SNVs, or more relative to a reference set of sequences. In particular embodiments, a set of genetic profiles can include 30 SNVs relative to a reference set of sequences. In particular embodiments, a reference set of sequences can be generated from a control or reference cell following the single cell analysis methods described herein. Particular embodiments of a control or reference cell include a cell of a known type (e.g., immune cell, cell particular to an organ or tissue) and known status (e.g., healthy, diseased, from a particular population of individuals), or a cell of a known type and unknown status. In particular embodiments, the lack of common members (e.g., genomic sequences, transcriptome sequences), either absolute lack or partial lack, can be used to distinguish a first set of genetic profiles for a given cell from a second set of genetic profiles for a given different cell. In particular embodiments, no members in a first set of genetic profiles intersect (are in common) with members in a second set of genetic profiles. In particular embodiments, a portion of members in a first set of genetic profiles intersect (are in common) with members in a second set of genetic profiles and a portion of members in the first set of genetic profiles do not intersect with a portion of members in the second set of genetic profiles.

(VIII) Computer Control Systems. The present disclosure provides computer control systems that are programmed to implement methods of the disclosure including spatial mapping based on spatial identifier type, amount, or density in relation to a point of application, nucleic acid sequencing methods, interpretation of nucleic acid sequencing data and analysis of cellular nucleic acids, such as RNA (e.g., mRNA), and characterization of cells from sequencing data. The computer system can be an electronic device of a user or a computer system that is remotely located with respect to the electronic device. The electronic device can be a mobile electronic device.

The computer system includes a central processing unit (CPU, also “processor” and “computer processor” herein), which can be a single core or multi core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network in some cases with the aid of the computer system can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that includes a user interface (UI) for providing, for example, results of spatial mapping analyses based on spatial identifier type, amount, or density in relation to a point of application, nucleic acid sequencing, analysis of nucleic acid sequencing data, characterization of nucleic acid sequencing samples, cell characterizations, etc. Examples of UI's include a graphical user interface (GUI) and web-based user interface.

Methods and systems of the present disclosure can be implemented by way of one or more algorithms. An algorithm can be implemented by way of software upon execution by the central processing unit. The algorithm can, for example, initiate spatial mapping analyses based on spatial identifier type, amount, or density in relation to a point of application, nucleic acid sequencing, process nucleic acid sequencing data, interpret nucleic acid sequencing results, characterize nucleic acid samples, characterize cells, etc.

(IX) Kits. Also disclosed herein are kits including one or more containers including one or more of components of the spatial localization and cell type identifying systems described herein. In particular embodiments, components can be included which are useful for labeling a biological sample to produce a localization gradient in the sample, for preparing or processing the biological sample (for instance, for disaggregation of cells in the tissue after they are labeled with spatial identifiers), for detecting or otherwise processing labels in disaggregated cells (including secondary detection molecules, or compounds useful in sequencing labeled nucleic acid molecules), and so forth. Also contemplated are kits that include one or more targeting components that permit at least one spatial identifier of the kit to be directed specifically to a target cell or category of cells, for instance an antibody or other targeting moiety useful for associating a label with target cells in the biological tissue which remains localized with the relevant cell(s) through disaggregation, thereby permitting identification of a subset of cells within the tissue through the presence (or greater abundance) of the targeted spatial identifier.

In particular embodiments, a kit can include a set of two or more spatial identifiers. For instance, such sets of spatial identifiers permit the detection and/or measurement (e.g., quantification) of two or more spatial identifiers in the same biological sample, without significant interference between the labels. Examples of such sets of spatial identifiers include fluorescent molecules with non-overlapping spectra, individual nucleotide sequences that can be separately detected through sequencing (such as highly-parallel sequencing technologies), and so forth.

Any functional component in a kit may be provided in premeasured amounts, though this is not required; and it is anticipated that certain kits will include more than functional amount, including for instance when the kit is used for a method requiring application of more than one functional amount.

Kits can also include a notice in the form prescribed by a governmental agency regulating the manufacture, use, or sale of pharmaceuticals or biological products, which notice reflects approval by the agency of manufacture, use, or sale for diagnostic or clinical use. The notice may state that the provided active ingredients can be administered to a biological sample. The kits can include further instructions for using the kit, for example, instructions regarding preparation of spatial identifiers or tissues for analysis; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as magnetic field generators, nanoparticles, oligonucleotides, sample appliers, syringes, ampules, software, sponges, imaging components, and the like. Variations in contents of any of the kits described herein can be made. The instructions of the kit will direct use of the components to effectuate a use described herein.

The Exemplary Embodiments and Examples below are included to demonstrate particular embodiments. Those of ordinary skill in the art will recognize in light of the present disclosure that many changes can be made to the specific embodiments disclosed herein and still obtain a like or similar result without departing from the spirit and scope of the disclosure.

### (X) Exemplary Embodiments

1. A method including:
   applying a spatial identifier to a location on a biological sample having a cell-type composition;
   disaggregating the biological sample;
   analyzing cells from the disaggregated biological sample for the presence of the spatial identifier;
   assigning a spatial location to each analyzed cell based on the presence, amount, or absence of the spatial identifier.
2. A method of embodiment 1, further including:
   associating a cellular label with each cell from the disaggregated biological sample;
   amplifying nucleic acid from each cell associated with a corresponding cellular label;
   sequencing the amplified nucleic acid from each cell to produce a corresponding cellular genetic profile;
   linking the amplified cellular label with the cellular genetic profile;
   identifying a cell type based on the cellular genetic profile;
   linking the cell type to the assigned spatial location based on the assigning and identifying.
3. A method of embodiment 1 or 2, wherein the cellular label is a genomic label.
4. A method of embodiment 1 or 2, wherein the cellular label is a transcriptional label.
5. A method of any one of embodiments 1-3, wherein the cellular genetic profile is a cellular genomic profile.
6. A method of any one of embodiments 1, 2, or 4, wherein the cellular genetic profile is a cellular transcriptome profile.
7. A method of any one of embodiments 1-6, wherein the location is recorded.
8. A method of any one of embodiments 1-7, wherein the biological sample is a tumor sample.
9. A method of any one of embodiments 1-8, wherein the cell-type composition is uniform.
10. A method of any one of embodiments 1-8, wherein the cell-type composition is heterogeneous.
11. A method of embodiment 10, wherein the heterogenous cell-type composition includes tumor-infiltrating lymphocytes.
12. A method of any one of embodiments 1-11, including applying at least 2 distinct spatial identifiers to locations on the biological sample, wherein each distinct spatial identifier is applied to a different location.
13. A method of embodiment 12, wherein the locations are recorded.
14. A method of any one of embodiments 1-13, wherein application of the spatial identifier creates a gradient of the spatial identifier within the biological sample.
15. A method of any one of embodiments 1-14, wherein the spatial identifier includes a nucleic acid barcode, a fluorescent molecule, a radioactive molecule, a chemiluminescent label, a spectral colorimetric label, a detectable tag, a fluorescence emitting metal, and/or a magnetic particle.
16. A method of any one of embodiments 1-15, wherein the spatial identifier is applied with an acoustic wave, heat, electromagnetic mobilization, electrophoresis, electroporation, injection, iontophoresis, magnetic charge, particle bombardment, pressure wave, sonophoresis, and/or ultrasound.
17. A method of any one of embodiments 1-16, wherein the spatial identifier is allowed to penetrate through simple diffusion.
18. A method of any one of embodiments 1-17, wherein the spatial identifier is attached to a binding agent, a bead, a nanoparticle, a magnetic particle, a polymer, a quantum dot, or a raman probe.
19. A method of embodiment 18, wherein the binding agent is an antibody.
20. A method of any one of embodiments 1-19, wherein the spatial identifier includes a nucleic acid barcode.
21. A method of embodiment 20, wherein the spatial identifier nucleic acid barcode includes sequencing adapters.
22. A method of embodiment 20 or 21, wherein the spatial identifier nucleic acid barcode includes a unique molecular identifier.
23. A method of any one of embodiments 20-22, wherein the spatial identifier nucleic acid barcode includes at least 10,000 identical copies of the nucleic acid barcode attached to a bead.
24. A method of any one of embodiments 1-23, wherein the spatial identifier includes a fluorescent molecule and cells from the biological sample are sorted by flow cytometry following disaggregation.
25. A method of any one of embodiments 1-24, wherein spatial identifiers are motivated through the biological sample with magnetic particles.
26. A method of any one of embodiments 1-25, wherein the spatial identifier is associated with a binding agent that binds a cell surface marker on a cell within the biological sample.
27. A method of embodiment 26, wherein the cell surface marker includes CD45 and/or CD102.
28. A method of embodiment 26 or 27, wherein the binding agent includes an anti-CD45 antibody or binding fragment thereof and/or an anti-CD102 antibody or binding fragment thereof.
29. A method of any one of embodiments 26-28, wherein the cell surface marker is expressed on tumor-infiltrating lymphocytes.
30. A method of embodiment 29, wherein the cell surface marker expressed on tumor-infiltrating lymphocytes includes CD3, CD4, and/or CD8.
31. A method of any one of embodiments 26-30, wherein the binding agent includes an anti-CD3 antibody or binding fragment thereof, an anti-CD4 antibody or binding fragment thereof, and/or an anti-CD8 antibody or binding fragment thereof.
32. A method of any one of embodiments 26-31, wherein the cell surface marker is expressed on cancer cells.
33. A method of embodiment 32, wherein the cell surface marker expressed on cancer cells is CD44, Prostate-Specific Membrane Antigen (PSMA), Wilms' Tumor 1 Antigen (WT1), Prostate Stem Cell Antigen (PSCA), Simian Vacuolating Virus 40 large T antigen (SV40 T), human epidermal growth factor 2 (HER2), receptor tyrosine kinase-like orphan receptor (ROR1), L1 cell adhesion molecule (L1-CAM), extracellular domain of MUC16 (MUC-CD), folate binding protein (folate receptor), Lewis Y carbohydrate antigen, or carboxy-anhydrase-IX (CAIX).
34. A method of any one of embodiments 26-33, wherein the binding agent includes an anti-CD44 antibody or binding fragment thereof, an anti-PSMA antibody or binding fragment thereof, an anti-WT1 antibody or binding fragment thereof, an anti-PSCA antibody or binding fragment thereof, an anti-SV40 T antibody or binding fragment thereof, an anti-HER2 antibody or binding fragment thereof, an anti-ROR1 antibody or binding fragment thereof, an anti-L1-CAM antibody or binding fragment thereof, an extracellular domain of MUC-CD antibody or binding fragment thereof, an anti-folate receptor antibody or binding fragment thereof, an anti-Lewis Y antibody or binding fragment thereof, an anti-mesothelin antibody or binding fragment thereof, or an anti-CAIX antibody or binding fragment thereof.
35. A method of any one of embodiments 1-34, wherein disaggregating the biological sample includes contacting the biological sample with hyaluronidase and/or thermolysin enzymes.
36. A method of any one of embodiments 1-35, including imaging the biological sample before disaggregation.
37. A method of any one of embodiments 1-36, including imaging the biological sample before and/or after application of spatial identifiers.
38. A method of any one of embodiments 1-37, including cutting the biological sample into portions before and/or after application of spatial identifiers and/or before and/or after imaging.
39. A method of any one of embodiments 1-38, including:
   cutting the biological sample after applying the spatial identifier;
   obtaining cells from the edge of the cut;
   assessing presence, amount, or concentration of spatial identifiers in or on the obtained cells; and
   using the assessed presence, amount, or concentration of spatial identifiers to provide a calibrating reference point for spatial mapping.
40. A method of any one of embodiments 1-38, including:
   obtaining cellular biopsies of the biological sample from known locations after applying the spatial identifier;
   assessing presence, amount, or concentration of spatial identifiers in or on the biopsied cells; and
   using the assessed presence, amount, or concentration of spatial identifiers to provide a calibrating reference point for spatial mapping.
41. A method of embodiment 39 or 40, wherein calibrated reference points are associated with one or more fiducial markers on an image.
42. A method of any one of embodiments 1-41, wherein assigning utilizes calibrated reference points or calibrated reference points associated with one or more fiducial markers on an image.
43. A method of any one of embodiments 1-42, including correlating location(s) in an image of the biological sample with spatial identifiers applied to the biological sample.
44. A method of any one of embodiments 1-43, wherein the assigning of a spatial location utilizes spatial reference data in a linear, nonlinear, logistic, polynomial, or exponential regression or fitting model.
45. A method of any one of embodiments 1-44, wherein the assigning of a spatial location utilizes computational modeling based on a neural network or a decision tree.
46. A method of any one of embodiments 2-45, wherein associating a cellular label with each cell from the disaggregated biological sample further includes partitioning disaggregated cells into single cell partitions wherein each partition includes
a single cell including a first set of polynucleotides; and
the corresponding cellular label including a nucleic acid barcode.
47. A method of any one of embodiments 2-46, wherein the cellular label is a genomic label.
48. A method of any one of embodiments 2-46, wherein the cellular label is a transcriptional label.
49. A method of any one of embodiments 2-48, wherein the cellular label further includes sequencing adapters.
50. A method of any one of embodiments 2-49, wherein the cellular label further includes a unique molecular identifier.
51. A method of embodiment 46, wherein the partitions are droplets.
52. A method of any one of embodiments 46-51, wherein the partition includes lysis reagents.
53. A method of any one of embodiments 46-52, wherein the partition includes nucleic acid amplification reagents.
54. A method of any one of embodiments 46-53, wherein the partition includes a polymerase.
55. A method of any one of embodiments 46-54, wherein the partition includes a template switching oligonucleotide.
56. A method of any one of embodiments 46-55, wherein the partition includes an endonuclease, DNA polymerase enzymes and dNTPs.
57. A method of any one of embodiments 46-56, wherein the partition includes reverse transcriptase enzymes, primers, and template switching oligonucleotides.
58. A method of any one of embodiments 46-57, including subjecting the first set of polynucleotides to nucleic acid amplification under conditions sufficient to generate a second set of polynucleotides, wherein a given polynucleotide of the second set of polynucleotides includes
   (i) a segment having a sequence of a polynucleotide of the first set of polynucleotides
   and (ii) a segment having a sequence of the cellular label nucleic acid barcode.
59. A method of embodiment 58, including generating a library of second sets of polynucleotides from a plurality of partitions, each partition including a partitioned cell of any one of embodiments 48-60.
60. A method of embodiment 59, including
(a) subjecting the library of second sets of polynucleotides to sequencing to yield sequencing reads, wherein each cellular label nucleic acid barcode sequence of the plurality of cellular label nucleic acid barcode sequences associate sequencing reads with each corresponding individual partitioned cell; and
(b) processing the sequencing reads associated with individual partitioned cells to generate
   (i) a first set of cellular genetic profiles corresponding to a first cell type and
   (ii) a second set of cellular genetic profiles corresponding to a second cell type, which first and second set of cellular genetic profiles differentiate a cell of the first cell type from a cell of the second cell type.
61. A method of embodiment 60, wherein the cellular genetic profiles are cellular genomic profiles.
62. A method of embodiment 60, wherein the cellular genetic profiles are cellular transcriptome profiles.
63. A method of any one of embodiments 60-62, wherein the first set of cellular genetic profiles and the second set of cellular genetic profiles include single nucleotide variants (SNVs).
64. A method of any one of embodiments 60-63, wherein each of the first and second set of cellular genetic profiles include at least 30 SNVs.
65. A method of any one of embodiments 60-64, wherein the first set of cellular genetic profiles and the second set of cellular genetic profiles do not intersect (do not share members).
66. A method of any one of embodiments 2-65, wherein the cellular label includes at least 10,000 identical nucleic acid barcodes.
67. A method of any one of embodiments 2-66, wherein the cellular label is associated with a bead.
68. A method of embodiment 67, wherein the bead is a gel bead.
69. A method of embodiment 67 or 68, wherein the cellular label is releasably attached to the bead.
70. A method of embodiment 69, including applying a stimulus to release the cellular label from the bead.
71. A method of embodiment 70, wherein the stimulus is a photo-stimulus, a thermal stimulus, or a chemical stimulus.
72. A method of any one of embodiments 2-71, wherein the sequencing includes high-throughput single cell transcriptomics.
73. A method of any one of embodiments 2-72, wherein the sequencing includes single-cell RNA sequencing (scRNA-seq).
74. A method including:
   applying a spatial identifier nucleic acid barcode to a location on a biological sample having a cell-type composition;
   disaggregating the biological sample into a suspension of cells; and
   separating the cells into single cell partitions wherein each partition includes (i) a first set of polynucleotides including the partitioned cell's endogenous genome and optionally a spatial identifier nucleic acid barcode; and (ii) a cellular label nucleic acid barcode that is different from the spatial identifier nucleic acid barcode.
75. A method of embodiment 74, wherein the cellular label is a genomic label.
76. A method of embodiment 74, wherein the cellular label is a transcriptional label.
77. A method of any one of embodiments 74-76, wherein the location is recorded.
78. A method of any one of embodiments 74-77, wherein the cell-type composition is uniform.
79. A method of any one of embodiments 74-77, wherein the cell-type composition is heterogeneous.
80. A method of any one of embodiments 74-79, wherein the spatial identifier nucleic acid barcode includes at least 10,000 identical spatial identifier nucleic acid barcodes and the cellular label nucleic acid barcode includes at least 10,000 identical cellular label nucleic acid barcodes.
81. A method of any one of embodiments 74-80, wherein the spatial identifier nucleic acid barcode is associated with a first bead and the cellular label nucleic acid barcode is associated with a second bead.
82. A method of embodiment 81, wherein the first bead and/or the second bead is a gel bead.
83. A method of embodiment 81 or 82, wherein the spatial identifier nucleic acid barcode and the cellular label nucleic acid barcode are releasably attached to their respective beads.
84. A method of embodiment 83, including applying a stimulus to release the spatial identifier nucleic acid barcode from its bead.
85. A method of embodiment 83 or 84, including applying a stimulus to release the cellular label nucleic acid barcode from its bead.
86. A method of embodiment 84 or 85, wherein the stimulus that releases the spatial identifier nucleic acid barcode from its bead is different from the stimulus that releases the cellular label nucleic acid barcode from its bead.
87. The method of embodiment 84 or 85, wherein the stimulus that releases the spatial identifier nucleic acid barcode from its bead is the same as the stimulus that releases the cellular label nucleic acid barcode from its bead.
88. A method of any one of embodiments 84-87, wherein the stimulus is a photo-stimulus, a thermal stimulus, and/or a chemical stimulus.
89. A method of any one of embodiments 74-88, wherein the spatial identifier nucleic acid barcode and the cellular label nucleic acid barcode are functionalized with sequencing adapters.
90. A method of any one of embodiments 74-89, wherein the spatial identifier nucleic acid barcode and/or the cellular label nucleic acid barcode includes a unique molecular identifier.
91. A method of any one of embodiments 74-90, wherein the spatial identifier is attached to a binding agent, a bead, a nanoparticle, a magnetic particle, a polymer, a quantum dot, or a raman probe.
92. A method of embodiment 91, wherein the binding agent is an antibody.
93. A method of any one of embodiments 74-92, wherein the spatial identifier is associated with a binding agent that binds a cell surface marker on a cell within the biological sample.
94. A method of embodiment 93, wherein the cell surface marker includes CD45 and/or CD102.
95. A method of embodiment 93 or 94, wherein the binding agent includes an anti-CD45 antibody or binding fragment thereof and/or an anti-CD102 antibody or binding fragment thereof.
96. A method of any one of embodiments 74-95, wherein disaggregating the biological sample includes contacting the biological sample with hyaluronidase and/or thermolysin enzymes.
97. A method of any one of embodiments 74-96, wherein the partitions are droplets.
98. A method of any one of embodiments 74-97, wherein the partition includes nucleic acid amplification reagents.
99. A method of any one of embodiments 74-98, wherein the partition includes a polymerase.
100. A method of any one of embodiments 74-99, wherein the partition includes a template switching oligonucleotide.
101. A method of any one of embodiments 74-100, wherein the partition includes an endonuclease, DNA polymerase enzymes and dNTPs.
102. A method of any one of embodiments 74-101, wherein the partition includes reverse transcriptase enzymes, primers, and template switching oligonucleotides.
103. A method of any one of embodiments 74-102, including subjecting the first set of polynucleotides to nucleic acid amplification under conditions sufficient to generate a second set of polynucleotides, wherein a given polynucleotide of the second set of polynucleotides includes
   (i) a segment having a sequence of a polynucleotide of the first set of polynucleotides;
   and (ii) a segment having a sequence of a cellular label nucleic acid barcode;
   and/or
   an amplified spatial identifier nucleic acid barcode.
104. A method of embodiment 103, including generating a library of second sets of polynucleotides from a plurality of partitions, each partition including a partitioned cell of any one of embodiments 74-103.
105. A method of embodiment 103 or 104, including
subjecting the library of second sets of polynucleotides to sequencing to yield sequencing reads, wherein
presence, amount, or type of spatial identifier nucleic acid barcode sequences allow assignment of a spatial location of the partitioned cell before biological sample disaggregation; and
cellular label nucleic acid barcode sequences of the plurality of cellular label nucleic acid barcode sequences associate sequencing reads with individual partitioned cells.
106.A method of embodiment 105, including processing the sequencing reads associated with individual partitioned cells to assign spatial location and identify cell type of individually partitioned cells.
107.A method of embodiment 106, wherein assigning of a spatial location utilizes calibrated reference points.
108.A method of embodiment 106 or 107, wherein assigning of a spatial location utilizes calibrated reference points that are associated with one or more fiducial markers on an image.
109.A method of any one of embodiments 106-108, wherein assigning of a spatial location utilizes correlating location(s) in an image of the biological sample with spatial identifier nucleic acid barcodes introduced to the biological sample.
110.A method of any one of embodiments 106-109, wherein assigning of a spatial location utilizes spatial reference data in a linear, nonlinear, logistic, polynomial, or exponential regression or fitting model.
111.A method of any one of embodiments 106-110, wherein assigning of a spatial location utilizes computational modeling based on a neural network or a decision tree.
112.A method of any one of embodiments 106-111, wherein the cell type of individually partitioned cells is identified utilizing a first set of cellular genetic profiles and a second set of cellular genetic profiles, each set including single nucleotide variants (SNVs).
113.A method of embodiment 112, wherein each of the first and second set of cellular genetic profiles includes at least 30 SNVs.
114.A method of embodiment 112 or 113, wherein the first set of genetic profiles and the second set of genetic profiles do not intersect (do not share members).
115.A method including reverse transcribing nucleic acid within a plurality of single cell partitions, wherein the nucleic acid within each partition includes (i) nucleic acid endogenous to the cell within the partition; (ii) a spatial identifier nucleic acid barcode; and (iii) a transcriptional label nucleic acid barcode;
generating a library of reverse transcribed nucleic acid from the plurality of single cell partitions; sequencing the generated library;
processing the sequencing data to enable automated cell clustering; and
assigning a spatial location and a cell type to cell clusters based on the reverse transcribed endogenous nucleic acid, the spatial identifier nucleic acid barcode; and the transcriptional label nucleic acid barcode.
116.A kit including components to practice a method of any of the preceding embodiments.
117.A kit of embodiment 116, including one or more of spatial identifiers, cellular labels, genomic labels, transcriptional labels, reagents for tissue disaggregation, beads, nanoparticles, magnetic particles, polymers, quantum dots, raman probes, sequencing adapters, unique molecular identifiers, binding agents (e.g., nucleic-acid bound binding agents), tissue cutting tools, tissue processing components, lysis reagents, nucleic acid amplification reagents, polymerases, template switching oligonucleotides, endonucleases, DNA polymerase enzymes, dNTPs, reverse transcriptase enzymes, primers, and information regarding genetic profiles to identify cell types.
118.A kit of embodiment 116 or 117, wherein spatial identifiers include one or more of nucleic acid barcodes, fluorescent molecules, radioactive molecules, chemiluminescent labels, spectral colorimetric labels, detectable tags, fluorescence emitting metals, and/or magnetic particles.
119.A kit of embodiment 117 or 118, wherein binding agents include one or more of anti-CD3 antibodies or binding fragments thereof, anti-CD4 antibodies or binding fragments thereof, anti-CD8 antibodies or binding fragments thereof, anti-CD44 antibodies or binding fragments thereof, anti-PSMA antibodies or binding fragments thereof, anti-WT1 antibodies or binding fragments thereof, anti-PSCA antibodies or binding fragments thereof, anti-SV40 T antibodies or binding fragments thereof, anti-HER2 antibodies or binding fragments thereof, anti-ROR1 antibodies or binding fragments thereof, anti-L1-CAM antibodies or binding fragments thereof, anti-extracellular domain of MUC-CD antibodies or binding fragments thereof, anti-folate receptor antibodies or binding fragments thereof, anti-Lewis Y antibodies or binding fragments thereof, anti-mesothelin antibodies or binding fragments thereof, and/or anti-CAIX antibodies or binding fragments thereof.

(XI) Examples. Example 1. There are myriad instances of rare cell types found within heterogenous tissues. By way of example, tumor-infiltrating immune cells such as tumor-infiltrating leukocytes (TILs) are a rare cell type that is found within tumor tissue, including non-small cell lung cancer. The methods and systems described in this Example can be used to characterize the presence, arrangement, distribution, and quantity of TILs within a tumor sample. This map information can be used to assess the clinical utility of characterizing these cells in relation to their position in the tumor.

A tumor sample is obtained, labeled with spatial identifiers, dissociated, and processed in accord with published methods described, for example, in Zheng et al. (Massively parallel digital transcriptional profiling of single cells. Nat Commun. 8:14049, 2017. doi:10.1038/ncomms14049). Briefly, this is a droplet-based system that enables 3′ messenger RNA (mRNA) digital counting of thousands of single cells. 50% of cells loaded into the system can be captured into droplets, and several (in some cases, up to eight) samples can be processed in parallel per run. Reverse transcription takes place inside each droplet, and barcoded complementary DNAs (cDNAs) are amplified in bulk. The resulting libraries then undergo Illumina or similar short-read sequencing. An analysis pipeline, such as Cell Ranger, is used to process the sequencing data and enables automated cell clustering. This results in the sequence of one (or more) selected target sequence(s) from each of the dissociated cells which are analyzed.

Computational deconvolution of the type and/or amount of individual spatial identifiers in disaggregated cells is used for spatial mapping of individual cells to their position within the original tissue sample. For example, cells that share similar spatial-identifier distributions (e.g., similar numbers and distributions of spatial identifiers, such as barcodes) will be spatially associated with one another. Pairwise comparisons among cells and their barcodes allows building of a 3D (or 2D, without loss of generality) spatial model of position.

In some examples, spatial-reference data such as described above is used in determining the 3D spatial model of position. In an illustrative example, the spatial-reference data can include a first coordinate, an associated first spatial-identifier distribution, a second coordinate, and an associated second spatial-identifier distribution. The first and second coordinates can be different. Each spatial-identifier distribution can include a respective concentration of a particular spatial identifier. Linear regression can be used based on the spatial-reference data [(concentration 1, coordinates 1), (concentration 2, coordinates 2)] to determine a linear function from concentration of the spatial identifier to position. Then, for any disaggregated cell, the concentration of the particular spatial identifier can be mapped through the linear function to determine the estimated coordinates of that cell's location in the labeled biological sample.

This example is illustrative and not limiting. Regression or other fitting techniques can be used to fit functions from any number of independent variables (e.g., one per spatial identifier) to any number of dependent variables (e.g., 1-3 dependent variables, representing respective coordinates in a 3D space). Regression or fitting models that can be used can include linear, nonlinear, logistic, polynomial, exponential, or other models. In other examples, the spatial-reference data can be fit to a predetermined basis, e.g., of wavelets, chirplets, or harmonic functions such as spherical harmonic functions. In some examples, fitting can be performed by gradient descent on an error metric that combines terms from each of the spatial identifiers, or more than one of the spatial identifiers, to balance error of a predetermined fitting model across the spatial identifiers.

In still further examples, multiple regression or fitting models can be determined. For example, three spatial identifiers can be used, each injected or otherwise added to the biological sample along one of three orthogonal axes in a coordinate system having an origin within the biological sample. Three fits can be performed: one to map concentration of the first identifier to the X coordinate, one to map concentration of the second identifier to the Y coordinate, and one to map concentration of the third identifier to the Z coordinate.

In still further examples, multiple spatial identifiers can be used on each axis. For example, two different spatial identifiers can be added to the biological sample on the +X and -X axes, respectively. A fit can then be determined from the concentrations of those two values to the X coordinate. This can improve coordinate accuracy in the case of nonuniform diffusion of a spatial identifier along the ±X axis.

In yet further examples, models mapping spatial-identifier distributions to coordinates, in any of the combinations of identifiers and coordinates above or in other combinations, can be determined using computational models such as neural networks, decision trees, or other machine-learning models. The spatial-reference data can be used as training data, or can be divided into training, test, or validation datasets. The training data can be used to train a computational model (CM) to take spatial-identifier distributions as inputs and produce coordinates as outputs.

In various examples, the CMs may include one or more regression models, e.g., polynomial and/or logistic regression models; classifiers such as binary classifiers; decision trees, e.g., boosted decision trees, configured for, e.g., classification or regression; and/or artificial neurons, e.g., interconnected to form a multilayer perceptron or other neural network. A decision tree can include, e.g., parameters defining hierarchical splits of a feature space into a plurality of regions. A decision tree can further include associated classes, values, or regression parameters associated with the regions. A neural network (NN) can have none, at least one, or at least two hidden layers. NNs having multiple hidden layers are referred to as deep neural networks (DNNs).

In some examples, CMs can include one or more recurrent computational models (RCMs). An RCM can include artificial neurons interconnected so that the output of a first unit can serve as a later input to the first unit and/or to another unit not in the layer immediately following the layer containing the first unit. Examples include Elman networks in which the outputs of hidden-layer artificial neurons are fed back to those neurons via memory cells, and Jordan networks, in which the outputs of output-layer artificial neurons are fed back via the memory cells. In some examples, an RCM can include one or more long short-term memory (LSTM) units. The computational model(s) can include, e.g., one or more DNNs, RCMs such as recurrent neural networks (RNNs), deep RNNs (DRNNs), Q-learning networks (QNs) and/or deep Q-learning networks (DQNs).

At least one of the computational model(s) can include, e.g., activation weights, functions, and/or thresholds for artificial neurons and/or other computational units (e.g., LSTM units) of one or more neural networks; coefficients of learned ranking functions, e.g., polynomial functions; and/or parameters of decision trees and/or other classifiers, in some nonlimiting examples. These are referred to individually or collectively as “parameters” herein.

A modeling engine can be configured to determine CMs, e.g., to apply NN-training techniques to determine neuron parameters of artificial neurons in the CMs. For example, modeling engines can determine CMs using a reinforcement-learning update rule. The modeling engine can parallelize the training of the NNs and/or other determination algorithms for CMs across multiple processing units, e.g., cores of a multi-core processor and/or multiple general-purpose graphics processing units (GPGPUs). For example, multiple layers of DNNs may be processed in parallel on the multiple processing units. The modeling engine can train neural networks such as DNNs minibatch-based stochastic gradient descent (SGD). SGD can be parallelized along, e.g., model parameters, layers, and data (and combinations thereof). Other frameworks besides SGD can be used, e.g., minibatch non-stochastic gradient descent and/or other mathematical-optimization techniques. The modeling engine can determine CMs at least in part using an experience replay or “bag-of-transitions” reinforcement-learning update rule.

In some examples, CMs can be determined and/or adjusted using the Theano and/or scikit-learn packages for PYTHON, and/or another symbolic/numerical equation solver, e.g., implemented in C++, C#, MATLAB, Octave, and/or MATHEMATICA. For example, training techniques can be implemented using, and/or can include, invocation(s) of the scikit-learn “fit( )” function to fit, e.g., gradient-boosted regression trees, linear functions, or other functional forms to regression data. In some examples, Theano can be used to perform NN training, e.g., using the “grad( )” and “function( )” routines.

In some examples, a learning-step function can be given as input a randomly-selected minibatch of the training data at each call in order to determine and/or adjust the model according to stochastic gradient descent (SGD) techniques. In some examples, computational models can be determined or adjusted using SGD with momentum. Grid search can be used to select the learning rate for, e.g., NN training and/or other CM determination and/or adjustment. Alternatively, several candidate policies can be determined using various learning rates and the candidate policy best satisfying acceptance criteria, e.g., of accuracy and/or precision, can be selected.

In some examples, a CM can be determined using one or more minibatches of the training data, e.g., a plurality of minibatches. A CM can be determined in a stochastic manner, e.g., by selecting minibatches at random from logging data. In some examples, using stochastic techniques can provide improved speed of convergence and/or improved numerical stability of the CM-determination process. In some examples, minibatches can be processed in parallel to reduce training time.

Example 2. Cells labeled with spatial identifiers are dissociated and processed to detect single nucleotide polymorphisms (SNPs) with single cell RNA sequencing data (e.g., transcriptome). Identified SNPs distinguish and identify cell types.

A droplet based microfluidic system partitions cells of a cell sample into droplets including gel beads. Partitions, or droplets, including cells and gel beads preferably contain one cell and one gel bead, but in some cases can contain various numbers of cells and various numbers of gel beads (including no cells or no gel beads). Briefly, droplets including gel beads (sometimes referred to as a GEM), are formed in an 8-channel microfluidic chip that encapsulates single gel beads at an 80% fill rate. Cells are combined with reagents in one channel of a microfluidic chip and then with gel beads from another channel to form GEMs. Reverse transcription (RT) is performed inside each GEM. Following RT, cDNAs are pooled for amplification and library construction in bulk. Each gel bead is functionalized with barcoded oligonucleotides including: i) sequencing adapters and primers, ii) a 14 bp transcriptional label barcode drawn from 750,000 designed sequences to index GEMs, iii) a 10 bp randomer to index molecules (unique molecular identifier, UMI), and iv) a 30 bp oligo-dT to prime poly-adenylated RNA transcripts. Within each microfluidic channel, 100,000 GEMs are formed per 6 min run, encapsulating thousands of cells in GEMs. Cells are loaded at a limiting dilution to minimize co-occurrence of multiple cells in the same GEM.

After encapsulation, cells are lysed and poly-adenylated RNAs are reverse transcribed. Each cDNA molecule produced will contain a transcriptional label and shared barcode per GEM, and end with a template switching oligo at the 3′ end. Next, the droplets are broken and barcoded cDNA is pooled for PCR amplification. Primers complementary to the switch oligos and sequencing adapters are used. Finally, amplified cDNAs are sheared, and adapter and sample indices are incorporated into finished libraries which are compatible with next-generation short-read sequencing. Read1 contains the cDNA insert while Read2 captures the transcriptional label. Index reads contain the sample indices and cell barcodes respectively. The approach enables parallel capture of thousands of cells in each of the 8 channels for scRNA-seq analysis.

Example 3. This Example shows technical validation data on using tagged antibodies to resolve spatial location. Briefly, fluorescently labeled anti-CD45 and anti-CD102 antibodies were diffused through a tissue slice. The tissue was dissociated according to the following protocol. The tissue was incubated in 300 μL serum-free Dulbecco's Modified Eagle Media (DMEM) containing hyaluronidase at 100 μg/mL (1 mg per 10 mL serum-free media) +3 pl Iiberase™ TL (low Thermolysin concentration, Sigma Millipore, Burlington, Mass.) for 15 min in a 24-well plate. Two μl DNAse I (4800 units/mL; 5 units/μL) was added to the media for another 15 min incubation. The cells of the tissue were resuspended every 5-10 min and observed under the microscope for dissociation. The dissociation was stopped with 1-2 mL of complete DMEM, and the entire solution was filtered with a 70 μm filter. The filtered solution was spun at 1500rpm for 5 min, the supernatant was discarded, and the cells resuspended in complete DMEM or 1× phosphate-buffered saline (PBS), depending on downstream application. A cell count was performed.

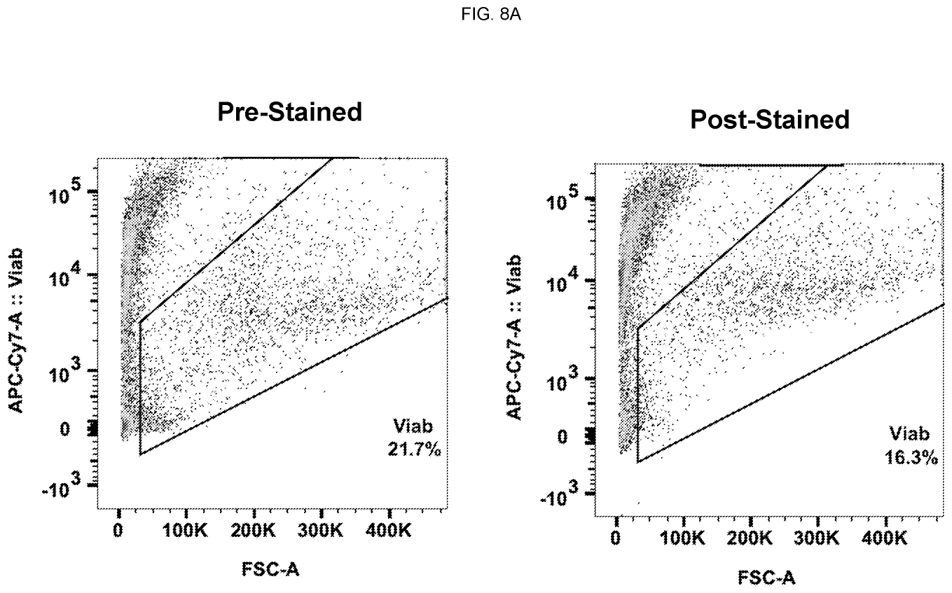

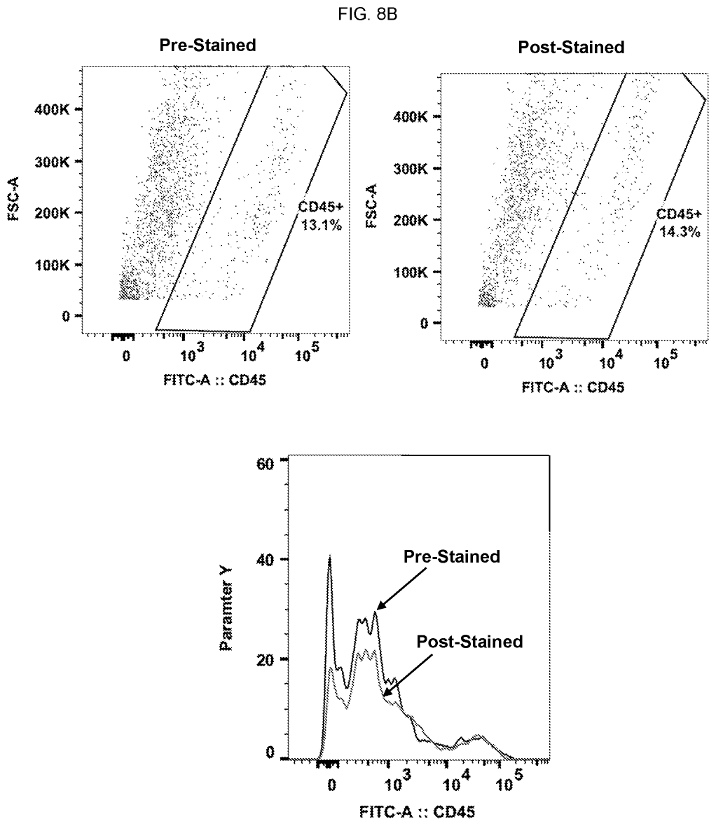

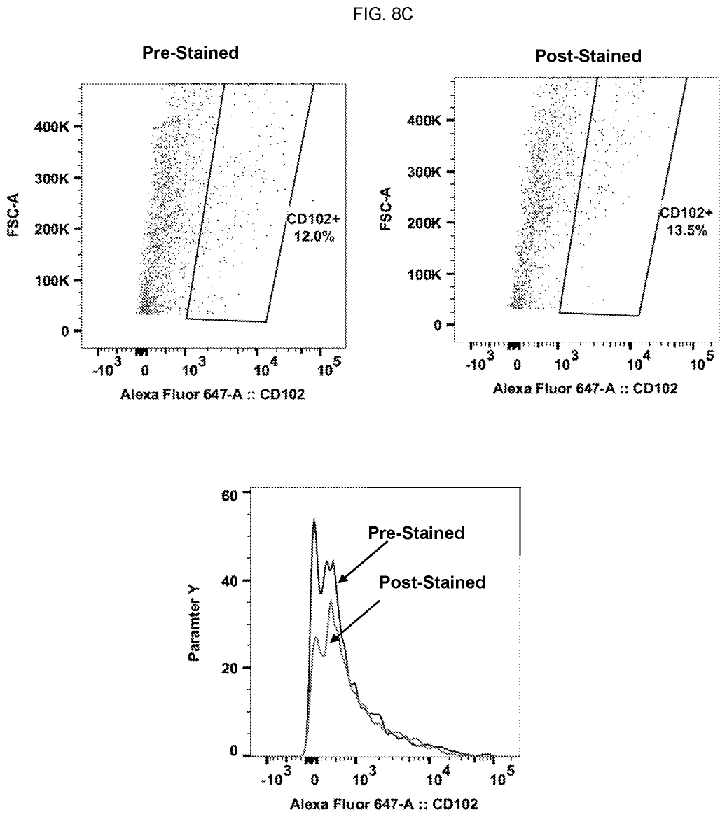

The percentage of CD45 and CD102 labeled cells were compared by flow cytometry resolved from a tissue slice labeled with the antibodies prior to (pre-stained) and labeled with the antibodies after (post-stained) creating a single cell suspension (FIGS. 8A-8C). FIG. 8A shows that the percentage of viable cells pre-stained (21.7%) and post-stained (16.3%) were similar. FIG. 8B shows that the percentage of CD45+ cells pre-stained (13.1%) and post-stained (14.3%) were similar. The median fluorescein isothiocyanate (FITC) intensity was 625 for viable post-stained cells and 517 for pre-stained viable cells (histogram of FIG. 8B). FIG. 8C shows that the percentage of CD102+ cells pre-stained (12.0%) and post-stained (13.5%) were similar. The median Alexa Fluor® 647 intensity was 596 for viable post-stained cells and 478 for pre-stained viable cells (histogram of FIG. 8C). Therefore, a tissue can be stained with fluorescently labeled antibodies binding to cell surface markers, and the labeled tissue can be dissociated to create a single cell suspension without negatively affecting the fluorescence signals from the antibodies or the number of cells. This study shows that antibodies including a spatial identifier can be used to label a tissue prior to dissociation, and the antibodies including a spatial identifier are not destroyed or removed by the dissociation process.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Eds. Attwood T et al., Oxford University Press, Oxford, 2006).

1. A method comprising:
applying a spatial identifier comprising a nucleic acid barcode to a location on a biological sample having a cell-type composition, wherein the spatial identifier is attached to a binding agent that binds a cell surface marker on a cell within the biological sample;
disaggregating the biological sample;
analyzing cells from the disaggregated biological sample for the presence of the spatial identifier nucleic acid barcode;
assigning a spatial location to each analyzed cell based on the presence, amount, or absence of the spatial identifier nucleic acid barcode;
associating a transcriptional label with each cell from the disaggregated biological sample;
amplifying nucleic acid from each cell associated with a corresponding transcriptional label;
sequencing the amplified nucleic acid from each cell to produce a corresponding cellular transcriptome profile;
linking the amplified transcriptional label with the cellular transcriptome profile;
identifying a cell type based on the cellular transcriptome profile;
linking the cell type to the assigned spatial location based on the assigning and identifying.

2. The method of claim 1, wherein the cell surface marker comprises CD45 and/or CD102.

3. The method of claim 1, wherein the binding agent comprises an anti-CD45 antibody or binding fragment thereof and/or an anti-CD102 antibody or binding fragment thereof.

4. The method of claim 1, wherein the binding agent comprises a fluorophore.

5. The method of claim 1, wherein disaggregating the biological sample comprises contacting the biological sample with hyaluronidase and/or thermolysin enzymes.

6. A method comprising:
applying a spatial identifier to a location on a biological sample having a cell-type composition;
disaggregating the biological sample;
analyzing cells from the disaggregated biological sample for the presence of the spatial identifier;
assigning a spatial location to each analyzed cell based on the presence, amount, or absence of the spatial identifier;
associating a transcriptional label with each cell from the disaggregated biological sample;
amplifying nucleic acid from each cell associated with a corresponding transcriptional label;
sequencing the amplified nucleic acid from each cell to produce a corresponding cellular transcriptome profile;
linking the amplified transcriptional label with the cellular transcriptome profile;
identifying a cell type based on the cellular transcriptome profile;
linking the cell type to the assigned spatial location based on the assigning and identifying.

7. The method of claim 6, wherein the location is recorded.

8. The method of claim 6, wherein the biological sample is a tumor sample.

9. The method of claim 6, wherein the cell-type composition is uniform.

10. The method of claim 6, wherein the cell-type composition is heterogeneous.

11. The method of claim 10, wherein the heterogenous cell-type composition comprises tumor-infiltrating lymphocytes.

12. The method of claim 6, comprising applying at least 2 distinct spatial identifiers to locations on the biological sample, wherein each distinct spatial identifier is applied to a different location.

13. The method of claim 12, wherein the locations are recorded.

14. The method of claim 6, wherein application of the spatial identifier creates a gradient of the spatial identifier within the biological sample.

15. The method of claim 6, wherein the spatial identifier comprises a nucleic acid barcode, a fluorescent molecule, a radioactive molecule, a chemiluminescent label, a spectral colorimetric label, a detectable tag, a fluorescence emitting metal, and/or a magnetic particle.

16. The method of claim 6, wherein the spatial identifier is applied with an acoustic wave, heat, electromagnetic mobilization, electrophoresis, electroporation, injection, iontophoresis, magnetic charge, particle bombardment, pressure wave, sonophoresis, and/or ultrasound.

17. The method of claim 6, wherein the spatial identifier is allowed to penetrate through simple diffusion.

18. The method of claim 6, wherein the spatial identifier is attached to a binding agent, a bead, a nanoparticle, a magnetic particle, a polymer, a quantum dot, or a raman probe.

19. The method of claim 18, wherein the binding agent is an antibody.

20. The method of claim 6, wherein the spatial identifier comprises a nucleic acid barcode.

21. The method of claim 20, wherein the spatial identifier nucleic acid barcode comprises sequencing adapters.

22. The method of claim 20, wherein the spatial identifier nucleic acid barcode comprises a unique molecular identifier.

23. The method of claim 20, wherein the spatial identifier nucleic acid barcode comprises at least 10,000 identical copies of the nucleic acid barcode attached to a bead.

24. The method of claim 6, wherein the spatial identifier is a fluorescent molecule and cells from the biological sample are sorted by flow cytometry following disaggregation.

25. The method of claim 6, wherein spatial identifiers are motivated through the biological sample with magnetic particles.

26. The method of claim 6, wherein the spatial identifier is associated with a binding agent that binds a cell surface marker on a cell within the biological sample.

27. The method of claim 26, wherein the cell surface marker comprises CD45 and/or CD102.

28. The method of claim 27, wherein the binding agent comprises an anti-CD45 antibody or binding fragment thereof and/or an anti-CD102 antibody or binding fragment thereof.

29. The method of claim 26, wherein the cell surface marker is expressed on tumor-infiltrating lymphocytes.

30. The method of claim 29, wherein the cell surface marker expressed on tumor-infiltrating lymphocytes comprises CD3, CD4, and/or CD8.

31. The method of claim 26, wherein the binding agent comprises an anti-CD3 antibody or binding fragment thereof, an anti-CD4 antibody or binding fragment thereof, and/or an anti-CD8 antibody or binding fragment thereof.

32. The method of claim 26, wherein the cell surface marker is expressed on cancer cells.

33. The method of claim 32, wherein the cell surface marker expressed on cancer cells is CD44, Prostate-Specific Membrane Antigen (PSMA), Wilms' Tumor 1 Antigen (WT1), Prostate Stem Cell Antigen (PSCA), Simian Vacuolating Virus 40 large T antigen (SV40 T), human epidermal growth factor 2 (HER2), receptor tyrosine kinase-like orphan receptor (ROR1), L1 cell adhesion molecule (L1-CAM), extracellular domain of MUC16 (MUC-CD), folate binding protein (folate receptor), Lewis Y carbohydrate antigen, or carboxy-anhydrase-IX (CAIX).

34. The method of claim 26, wherein the binding agent comprises an anti-CD44 antibody or binding fragment thereof, an anti-PSMA antibody or binding fragment thereof, an anti-WT1 antibody or binding fragment thereof, an anti-PSCA antibody or binding fragment thereof, an anti-SV40 T antibody or binding fragment thereof, an anti-HER2 antibody or binding fragment thereof, an anti-ROR1 antibody or binding fragment thereof, an anti-L1-CAM antibody or binding fragment thereof, an extracellular domain of MUC-CD antibody or binding fragment thereof, an anti-folate receptor antibody or binding fragment thereof, an anti-Lewis Y antibody or binding fragment thereof, an anti-mesothelin antibody or binding fragment thereof, or an anti-CAIX antibody or binding fragment thereof.

35. The method of claim 6, wherein disaggregating the biological sample comprises contacting the biological sample with hyaluronidase and/or thermolysin enzymes.

36. The method of claim 6, comprising imaging the biological sample before disaggregation.

37. The method of claim 6, comprising imaging the biological sample before and/or after application of spatial identifiers.

38. The method of claim 6, comprising cutting the biological sample into portions before and/or after application of spatial identifiers and/or before and/or after imaging.

39. The method of claim 6, comprising:
cutting the biological sample after applying the spatial identifier;
obtaining cells from the edge of the cut;
assessing presence, amount, or concentration of spatial identifiers in or on the obtained cells; and
using the assessed presence, amount, or concentration of spatial identifiers to provide a calibrating reference point for spatial mapping.

40. The method of claim 6, comprising:
obtaining cellular biopsies of the biological sample from known locations after applying the spatial identifier;
assessing presence, amount, or concentration of spatial identifiers in or on the biopsied cells; and
using the assessed presence, amount, or concentration of spatial identifiers to provide a calibrating reference point for spatial mapping.

41. The method of claim 39 or 40, wherein calibrated reference points are associated with one or more fiducial markers on an image.

42. The method of claim 6, wherein assigning utilizes calibrated reference points or calibrated reference points associated with one or more fiducial markers on an image.

43. The method of claim 6, comprising correlating location(s) in an image of the biological sample with spatial identifiers applied to the biological sample.

44. The method of claim 6, wherein the assigning of a spatial location utilizes spatial reference data in a linear, nonlinear, logistic, polynomial, or exponential regression or fitting model.

45. The method of claim 6, wherein the assigning of a spatial location utilizes computational modeling based on a neural network or a decision tree.

46. The method of claim 6, wherein associating a transcriptional label with each cell from the disaggregated biological sample further comprises partitioning disaggregated cells into single cell partitions wherein each partition comprises
a single cell comprising a first set of polynucleotides; and
the corresponding transcriptional label comprising a nucleic acid barcode.

47. The method of claim 46, wherein the partitions are droplets.

48. The method of claim 46, wherein the transcriptional label further comprises sequencing adapters.

49. The method of claim 46, wherein the transcriptional label further comprises a unique molecular identifier.

50. The method of claim 46, wherein the partition comprises lysis reagents.

51. The method of claim 46, wherein the partition comprises nucleic acid amplification reagents.

52. The method of claim 46, wherein the partition comprises a polymerase.

53. The method of claim 46, wherein the partition comprises a template switching oligonucleotide.

54. The method of claim 46, wherein the partition comprises an endonuclease, DNA polymerase enzymes and dNTPs.

55. The method of claim 46, wherein the partition comprises reverse transcriptase enzymes, primers, and template switching oligonucleotides.

56. The method of claim 46, comprising
subjecting the first set of polynucleotides to nucleic acid amplification under conditions sufficient to generate a second set of polynucleotides, wherein a given polynucleotide of the second set of polynucleotides comprises
(i) a segment having a sequence of a polynucleotide of the first set of polynucleotides
and (ii) a segment having a sequence of a transcriptional label nucleic acid barcode.

57. The method of claim 56, comprising generating a library of second sets of polynucleotides from a plurality of partitions, each partition comprising a partitioned cell of claim 48.

58. The method of claim 57, comprising
(a) subjecting the library of second sets of polynucleotides to sequencing to yield sequencing reads, wherein each transcriptional label nucleic acid barcode sequence of the plurality of transcriptional label nucleic acid barcode sequences associate sequencing reads with each corresponding individual partitioned cell; and
(b) processing the sequencing reads associated with individual partitioned cells to generate
(i) a first set of transcriptome profiles corresponding to a first cell type and
(ii) a second set of transcriptome profiles corresponding to a second cell type, which first and second set of transcriptome profiles differentiate a cell of the first cell type from a cell of the second cell type.

59. The method of claim 58, wherein the first set of transcriptome profiles and the second set of transcriptome profiles comprise single nucleotide variants (SNVs).

60. The method of claim 58, wherein each of the first and second set of transcriptome profiles comprise at least 30 SNVs.

61. The method of claim 58, wherein the first set of transcriptome profiles and the second set of transcriptome profiles do not intersect (do not share members).

62. The method of claim 6, wherein the transcriptional label is associated with a bead.

63. The method of claim 6, wherein the bead is a gel bead.

64. The method of claim 62, wherein the transcriptional label is releasably attached to the bead.

65. The method of claim 6, wherein the transcriptional label comprises at least 10,000 identical nucleic acid barcodes.

66. The method of claim 64, comprising applying a stimulus to release the transcriptional label from the bead.

67. The method of claim 66, wherein the stimulus is a photo-stimulus, a thermal stimulus, or a chemical stimulus.

68. The method of claim 6, wherein the sequencing comprises high-throughput single cell transcriptomics.

69. The method of claim 6, wherein the sequencing comprises single-cell RNA sequencing (scRNA-seq).

70. A method comprising:
applying a spatial identifier nucleic acid barcode to a location on a biological sample having a cell-type composition;
disaggregating the biological sample into a suspension of cells; and
separating the cells into single cell partitions wherein each partition comprises (i) a first set of polynucleotides comprising the partitioned cell's endogenous genome and optionally a spatial identifier nucleic acid barcode; and (ii) a transcriptional label nucleic acid barcode that is different from the spatial identifier nucleic acid barcode.

71. The method of claim 70, wherein the location is recorded.

72. The method of claim 70, wherein the cell-type composition is uniform.

73. The method of claim 70, wherein the cell-type composition is heterogeneous.

74. The method of claim 70, wherein the partitions are droplets.

75. The method of claim 70, wherein the spatial identifier nucleic acid barcode is associated with a first bead and the transcriptional label nucleic acid barcode is associated with a second bead.

76. The method of claim 75, wherein the first bead and/or the second bead is a gel bead.

77. The method of claim 75, wherein the spatial identifier nucleic acid barcode and the transcriptional label nucleic acid barcode are releasably attached to their respective beads.

78. The method of claim 70, wherein the spatial identifier nucleic acid barcode comprises at least 10,000 identical spatial identifier nucleic acid barcodes and the transcriptional label nucleic acid barcode comprises at least 10,000 identical transcriptional label nucleic acid barcodes.

79. The method of claim 78, comprising applying a stimulus to release the spatial identifier nucleic acid barcode from its bead.

80. The method of claim 78, comprising applying a stimulus to release the transcriptional label nucleic acid barcode from its bead.

81. The method of claim 79 or 80, wherein the stimulus that releases the spatial identifier nucleic acid barcode from its bead is different from the stimulus that releases the transcriptional label nucleic acid barcode from its bead.

82. The method of claim 79 or 80, wherein the stimulus that releases the spatial identifier nucleic acid barcode from its bead is the same as the stimulus that releases the transcriptional label nucleic acid barcode from its bead.

83. The method of claim 79 or 80, wherein the stimulus is a photo-stimulus, a thermal stimulus, or a chemical stimulus.

84. The method of claim 70, wherein the spatial identifier nucleic acid barcode and the transcriptional label nucleic acid barcode are functionalized with sequencing adapters.

85. The method of claim 70, wherein the spatial identifier nucleic acid barcode and/or the transcriptional label nucleic acid barcode comprises a unique molecular identifier.

86. The method of claim 70, wherein the partition comprises nucleic acid amplification reagents.

87. The method of claim 70, wherein the partition comprises a polymerase.

88. The method of claim 70, wherein the partition comprises a template switching oligonucleotide.

89. The method of claim 70, wherein the partition comprises an endonuclease, DNA polymerase enzymes and dNTPs.

90. The method of claim 70, wherein the partition comprises reverse transcriptase enzymes, primers, and template switching oligonucleotides.

91. The method of claim 70, comprising
subjecting the first set of polynucleotides to nucleic acid amplification under conditions sufficient to generate a second set of polynucleotides, wherein a given polynucleotide of the second set of polynucleotides comprises
(i) a segment having a sequence of a polynucleotide of the first set of polynucleotides;
and (ii) a segment having a sequence of a transcriptional label nucleic acid barcode;
and/or
an amplified spatial identifier nucleic acid barcode.

92. The method of claim 91, comprising generating a library of second sets of polynucleotides from a plurality of partitions, each partition comprising a partitioned cell of claim 66.

93. The method of claim 92, comprising
subjecting the library of second sets of polynucleotides to sequencing to yield sequencing reads, wherein
presence, amount, or type of spatial identifier nucleic acid barcode sequences allow assignment of a spatial location of the partitioned cell before biological sample disaggregation; and
transcriptional label nucleic acid barcode sequences of the plurality of transcriptional label nucleic acid barcode sequences associate sequencing reads with individual partitioned cells.

94. The method of claim 93, comprising processing the sequencing reads associated with individual partitioned cells to assign spatial location and identify cell type of individually partitioned cells.

95. The method of claim 94, wherein assigning of a spatial location utilizes calibrated reference points.

96. The method of claim 94, wherein assigning of a spatial location utilizes calibrated reference points that are associated with one or more fiducial markers on an image.

97. The method of claim 94, wherein assigning of a spatial location utilizes correlating location(s) in an image of the biological sample with spatial identifier nucleic acid barcodes introduced to the biological sample.

98. The method of claim 94, wherein assigning of a spatial location utilizes spatial reference data in a linear, nonlinear, logistic, polynomial, or exponential regression or fitting model.

99. The method of claim 94, wherein assigning of a spatial location utilizes computational modeling based on a neural network or a decision tree.

100. The method of claim 94, wherein the cell type of individually partitioned cells is identified utilizing a first set of genetic profiles and a second set of genetic profiles, each set comprising single nucleotide variants (SNVs).

101. The method of claim 100, wherein each of the first and second set of genetic profiles comprise at least 30 SNVs.

102. The method of claim 100, wherein the first set of genetic profiles and the second set of genetic profiles do not intersect (do not share members).

103. A method comprising reverse transcribing nucleic acid within a plurality of single cell partitions, wherein the nucleic acid within each partition comprises (i) nucleic acid endogenous to the cell within the partition; (ii) a spatial identifier nucleic acid barcode; and (iii) a transcriptional label nucleic acid barcode;
generating a library of reverse transcribed nucleic acid from the plurality of single cell partitions;
sequencing the generated library;
processing the sequencing data to enable automated cell clustering; and
assigning a spatial location and a cell type to cell clusters based on the reverse transcribed endogenous nucleic acid, the spatial identifier nucleic acid barcode; and the transcriptional label nucleic acid barcode.

104. A kit comprising components to practice a method of any of the preceding claims.

105. A kit of claim 104, comprising one or more of spatial identifiers, transcriptional labels, reagents for tissue disaggregation, beads, nanoparticles, magnetic particles, polymers, quantum dots, raman probes, sequencing adapters, unique molecular identifiers, binding agents, tissue cutting tools, tissue processing components, lysis reagents, nucleic acid amplification reagents, polymerases, template switching oligonucleotides, endonucleases, DNA polymerase enzymes, dNTPs, reverse transcriptase enzymes, primers, and information regarding genetic profiles to identify cell types.

106. A kit of claim 105, wherein spatial identifiers comprise one or more of nucleic acid barcodes, fluorescent molecules, radioactive molecules, chemiluminescent labels, spectral colorimetric labels, detectable tags, fluorescence emitting metals, and/or magnetic particles.

107. A kit of claim 105, wherein binding agents comprise one or more of anti-CD3 antibodies or binding fragments thereof, anti-CD4 antibodies or binding fragments thereof, anti-CD8 antibodies or binding fragments thereof, anti-CD44 antibodies or binding fragments thereof, anti-PSMA antibodies or binding fragments thereof, anti-WT1 antibodies or binding fragments thereof, anti-PSCA antibodies or binding fragments thereof, anti-SV40 T antibodies or binding fragments thereof, anti-HER2 antibodies or binding fragments thereof, anti-ROR1 antibodies or binding fragments thereof, anti-L1-CAM antibodies or binding fragments thereof, anti-extracellular domain of MUC (MUC-CD) antibodies or binding fragments thereof, anti-folate receptor antibodies or binding fragments thereof, anti-Lewis Y antibodies or binding fragments thereof, anti-mesothelin antibodies or binding fragments thereof, and/or anti-CAIX antibodies or binding fragments thereof.


SEQUENCE LISTING (15 sequences)

SEQ ID NO: 1 - DNA, 14 bp, Artificial Sequence
  exemplary PCR handle sequence
tcgtcggcagcgtc

SEQ ID NO: 2 - DNA, 15 bp, Artificial Sequence
  exemplary PCR handle sequence
gtctcgtgggctcgg

SEQ ID NO: 3 - DNA, 29 bp, Artificial Sequence
  exemplary PCR handle sequence
aatgatacggcgaccaccgagatctacac

SEQ ID NO: 4 - DNA, 24 bp, Artificial Sequence
  exemplary PCR handle sequence
caagcagaagacggcatacgagat

SEQ ID NO: 5 - DNA, 20 bp, Artificial Sequence
  exemplary PCR handle sequence
ccttggcacccgagaattcc

SEQ ID NO: 6 - protein, 8 aa, Artificial Sequence
  STREP tag II
WSHPQFEK

SEQ ID NO: 7 - protein, 8 aa, Artificial Sequence
  Flag tag
DYKDDDDK

SEQ ID NO: 8 - protein, 8 aa, Artificial Sequence
  Xpress tag
DLYDDDDK

SEQ ID NO: 9 - protein, 15 aa, Artificial Sequence
  Avi tag
GLNDIFEAQKIEWHE

SEQ ID NO: 10 - protein, 26 aa, Artificial Sequence
  Calmodulin tag
KRRWKKNFIAVSAANRFKKISSSGAL

SEQ ID NO: 11 - protein, 9 aa, Artificial Sequence
  Hemagglutinin (HA) tag
YPYDVPDYA

SEQ ID NO: 12 - protein, 10 aa, Artificial Sequence
  Myc tag
EQKLISEEDL

SEQ ID NO: 13 - protein, 13 aa, Artificial Sequence
  Softag 1
SLAELLNAGLGGS

SEQ ID NO: 14 - protein, 8 aa, Artificial Sequence
  Softag 3
TQDPSRVG

SEQ ID NO: 15 - protein, 14 aa, Artificial Sequence
  V5 tag
GKPIPNPLLGLDST